SEQUENCE LISTING (20 sequences)

SEQ ID NO: 1 - DNA, 5880 bp, Bifidobacterium bifidum
atgaaacatagagcgatgtcatcgcgtctgatgccactggtggcgtcctgcgcgacggtcggcatgctgctggccggactacctgtgtcggccgtcgcggtcggcacgacgagagcggcagcgtccgacgcctcgtcctccaccacagcaaccatcaccccctccgccgataccacgttgcagacatggacgagcgagaagaattcctcaatggcgtccaagccgtacatcggcacactgcaagggccctcgcaaggcgtgttcggcgagaagttcgagtccacggatgccgcggacaccaccgatctgaagaccggcctgctgacgttcgacctgagcgcctacgaccatgcccccgattccgcaacgttcgagatgacgtacctcggctaccgcggcaacccgacggccaccgacaccgacaccatcaaggtgacccccgtcgacaccaccgtgtgcaccaataacgccacagactgcggcgcgaatgtcgcgaccggcgcgaccaagccgaagttcagcatcaacgactcctcattcgtcgccgagtccaagccgttcgagtacggtacgacggtttacacgggcgacgccatcaccgtggttcccgccaataccaagaaggtcaccgtagatgtgaccgaaatcgtgcgccagcagttcgccgaaggcaagaaggtcatcaccctggccgtgggcgagaccaagaagaccgaggttcgtttcgccagttccgaaggcacgacgtccctgaacggcgcgaccgcagacatggctccgaagctgaccgtttccgtgtccaccaaggacgatctcaagccctccgccgacaccacgttgcaggcatgggccagcgagaagaacgagaagaagaacactgcggcctatgtcggcgcgctgcagccggaaggcgattacggcgacttcggtgagaagttcaagtccaccgacgtccacgatgtcacagacgccaagatgggtctgatgacgttcgacctgtccgattacaccgcggcgcccgagcactccatcctcaccttgacgtatctgggctacgccggtgcagacaagaccgccacggccaccgataaggtcaaggtggtcgctgttgacacgtcgcggtgcaccggcaccgctccctgcgacaccaacaatgccacgtgggcgaaccgcccggacttcgaggtgaccgataccacgaagaccgcgacgtcccatgcgttcgcttatggatctaagaagtattccgatggcatgaccgtcgaatcgggcaacgccaagaaggtcctgctcgacgtgtccgatgtcatcaaggcagagttcgccaagttcagcgccggcgccaccgagaagaagatcacgctggccctgggcgagctcaacaagtccgacatgcgtttcggcagcaaggaagtcacctcgctgaccggcgccaccgaagccatgcagccgaccttgtccgtcaccaagaagccgaaggcatacacgctgagcatcgaaggcccgaccaaggtcaagtaccagaagggcgaggcgttcgacaaggccggactcgtggtcaaggccaccagcacggctgacggcacggtcaagacgctgaccgaaggcaacggtgaggataactacaccatcgacaccagcgctttcgatagtgccagcatcggcgtataccctgttaccgtgaagtacaacaaggaccccgaaatcgccgcttcgttcaacgcctatgtcatcgccagtgtcgaggacggcggagacggcgacaccagcaaagacgactggctgtggtacaagcagcccgcgtcgcagaccgacgccaccgccaccgccggcggcaattacggcaaccccgacaacaaccgttggcagcagaccaccttgccgttcggcaacggcaagatcggcggcaccgtctggggcgaggtcagccgtgaacgcgtcaccttcaacgaggagacgctgtggaccggcggccccggatcctcgaccagctacaacggcggcaacaacgagaccaagggtcagaacggcgccacgctgcgcgcgctcaacaagcagctcgcgaacggcgccgagacggtcaatcccggcaacctgaccggcggcgagaacgcggccgagcagggcaactacctgaactggggcgacatctacctcgactacgggttcaacgatacgaccgtcaccgaataccgccgcgacctgaacctgagcaagggcaaggccgacgtcacgttcaagcatgacggcgtcacctacacgcgcgaatacttcgcgtcgaaccccgacaatgtcatggtcgcccgcctcacggccagcaaagccggcaagctgaacttcaacgtcagcatgccgaccaacacgaactactccaagaccggcgaaaccacgacggtcaagggtgacacgctcaccgtcaagggcgctctcggcaacaacggcctgctgtacaactcgcagatcaaggtcgtcctcgacaacggtgagggcacgctctccgaaggctccgacggcgcttcgctgaaggtctccgacgcgaaggcggtcacgctgtacatcgccgccgcgacggactacaagcagaagtatccgtcctaccgcaccggcgaaaccgccgccgaggtgaacacccgcgtcgccaaggtcgtgcaggacgccgccaacaagggctacaccgccgtcaagaaagcgcacatcgacgatcattccgccatctacgaccgcgtgaagatcgatttgggccagtccggccacagctccgacggcgccgtcgccaccgacgcgctgctcaaggcgtaccagagaggctccgcaaccaccgcgcagaagcgcgagctggagacgctggtgtacaagtacggccgctacttgaccatcggctcctcccgtgagaacagccagctgcccagcaacctgcagggcatctggtcggtcaccgcgggcgacaacgcccacggcaacacgccttggggctccgacttccacatgaacgtgaacctccagatgaactactggccgacctattcggccaacatgggagagctcgccgagccgctcatcgagtatgtggagggtctggtcaagcccggccgtgtgaccgccaaggtctacgcgggcgcggagacgacgaaccccgagaccacgccgatcggcgagggcgagggctacatggcccacaccgagaacaccgcctacggctggaccgcacccggtcaatcgttctcgtggggttggagcccggccgccgtgccgtggatcctgcagaacgtgtacgaggcgtacgagtactccggcgaccctgccctgcttgatcgcgtgtacgcgctgctcaaggaggaatcgcacttctacgtcaactacatgctgcacaaggccggctccagctccggtgaccgcctgactaccggcgtcgcgtactcgcccgaacagggcccgctgggcaccgacggcaacacgtacgagagctcgctcgtgtggcagatgctcaacgacgccatcgaggcggccaaggccaagggagatccggacggtctggtcggcaataccaccgactgctcggccgacaactgggccaagaatgacagcggcaacttcaccgatgcgaacgccaaccgttcctggagctgcgccaagagcctgctcaagccgatcgaggtcggcgactccggccagatcaaggaatggtacttcgaaggtgcgctcggcaagaagaaggatggatccaccatcagcggctaccaggcggacaaccagcaccgtcacatgtcccacctgctcggactgttccccggtgatttgatcaccatcgacaactccgagtacatggatgcggccaagacctcgctgaggtaccgctgcttcaagggcaacgtgctgcagtccaacaccggctgggccattggccagcgcatcaattcgtgggctcgcaccggcgacggcaacaccacgtaccagctggtcgagctgcagctcaagaacgcgatgtatgcaaacctgttcgattaccatgcgccgttccagatcgacggcaacttcggcaacacctccggtgtcgacgaaatgctgctgcagtccaactccaccttcaccgacaccgccggcaagaagtacgtgaactacacgaacatcctgcccgccctgcccgatgcctgggcgggcggctcggtgagcggcctcgtggcccgcggcaacttcaccgtcggcacgacatggaagaacggcaaggccaccgaagtcaggctgacctccaacaagggcaagcaggcggccgtcaagatcaccgccggcggcgcccagaactacgaggtcaagaacggtgacaccgccgtgaacgccaaggtcgtgaccaacgcggacggcgcctcgctgctcgtgttcgataccaccgcaggcaccacgtacacgatcacgaagaaggcgagcgccaacgtgcccgtcaccggcgtgaccgtgaccggcgccaacaccgccaccgcaggcgacaccgtcactcttacggctaccgtcgccccggccaatgcgaccgacaagtccgtcacctggtcgacctccgacgccgccgtagctacggtcaacgccaacggcgtggtgaccacgaagaaggccggcaaggtgaccatcaccgccacgtcgaacggcgacaagacgaagttcggttccatcgagatcaccgtctccgccgcgaccgtgcccgtcaccagcgtcaccgttgccggcgacgccgcgatgaccgtcgatggagagcagaccctgacggcgaccgtcgccccggccactgcgaccgacaagacggtcacgtggaagtcctccgacgccactgtggcgacggttgacgccaacggcaaggtcgtcgcgaagaaggccggcgaagtgacgatcaccgccacggccggtggcgtgtccggcacgctgaagatcacggtgagcgacaaggccccgaccgtcatcccggtccagtccgtgaccgtgacaggcaagcaggagctcgtcgaaggcgcctccacgaccctgacggcgaccgtcgccccggctgacgcgaccgacaagacggttacgtggaagtcgagcgacgagtccgtcgccacggtcgacaaggacggcgtcgtgaccgccaagaaggccggcacggtgaccatcaccgccacggccggtggcgtgtccggcacgctccacatcaccgtgacggccaagcccgtcgagaccgtccccgtcaccagcgtggaggtcaccgtcgaggccggcaccaccgtctccgtcggcaagacactccaggccaccgcgaccgtcaagcccggcaacgccaccaacaagaaggtgacgtggaagtcgagcgacgaatccatcgcgacggtcgacgccaacggcgtcatcaccgcgaagaaggccggcaaggtcgtcatcacggccacctcgaccgacggcacggacaagtccggcagcgtcgagatcaccgtcgtggatgagaccaagccgacgcccgaccacaagtccgtcaaggccgataccggcgacgtgaccgccggcaagaccggtacggtcaccgagccgaaggacgtggcgggctggaagagccgctccatcatcaagcaaggcaagctcggcaaggccgaaatcgccgacggcacgctcgtgtatgcggccggcgacaagaccggtgacgacagcttcgtcgtgcagtacacgatggccgacggcacggtcatcgacgtgacctacagcgtcacggtcaaggccgccgaaaccggcaagaacgacggcgacggcaagggcgacggtgtcgcgaagaccggcgccgccgtcggcgcgctcgccggcctcggcttgatgctgctcgccgtcggagtgagcgtggtgatgattcgccgcaagcactccgcctga

SEQ ID NO: 2 - protein, 1959 aa, Bifidobacterium bifidum
MKHRAMSSRLMPLVASCATVGMLLAGLPVSAVAVGTTRAAASDASSSTTATITPSADTTLQTWTSEKNSSMASKPYIGTLQGPSQGVFGEKFESTDAADTTDLKTGLLTFDLSAYDHAPDSATFEMTYLGYRGNPTATDTDTIKVTPVDTTVCTNNATDCGANVATGATKPKFSINDSSFVAESKPFEYGTTVYTGDAITVVPANTKKVTVDVTEIVRQQFAEGKKVITLAVGETKKTEVRFASSEGTTSLNGATADMAPKLTVSVSTKDDLKPSADTTLQAWASEKNEKKNTAAYVGALQPEGDYGDFGEKFKSTDVHDVTDAKMGLMTFDLSDYTAAPEHSILTLTYLGYAGADKTATATDKVKVVAVDTSRCTGTAPCDTNNATWANRPDFEVTDTTKTATSHAFAYGSKKYSDGMTVESGNAKKVLLDVSDVIKAEFAKFSAGATEKKITLALGELNKSDMRFGSKEVTSLTGATEAMQPTLSVTKKPKAYTLSIEGPTKVKYQKGEAFDKAGLVVKATSTADGTVKTLTEGNGEDNYTIDTSAFDSASIGVYPVTVKYNKDPEIAASFNAYVIASVEDGGDGDTSKDDWLWYKQPASQTDATATAGGNYGNPDNNRWQQTTLPFGNGKIGGTVWGEVSRERVTFNEETLWTGGPGSSTSYNGGNNETKGQNGATLRALNKQLANGAETVNPGNLTGGENAAEQGNYLNWGDIYLDYGFNDTTVTEYRRDLNLSKGKADVTFKHDGVTYTREYFASNPDNVMVARLTASKAGKLNFNVSMPTNTNYSKTGETTTVKGDTLTVKGALGNNGLLYNSQIKVVLDNGEGTLSEGSDGASLKVSDAKAVTLYIAAATDYKQKYPSYRTGETAAEVNTRVAKVVQDAANKGYTAVKKAHIDDHSAIYDRVKIDLGQSGHSSDGAVATDALLKAYQRGSATTAQKRELETLVYKYGRYLTIGSSRENSQLPSNLQGIWSVTAGDNAHGNTPWGSDFHMNVNLQMNYWPTYSANMGELAEPLIEYVEGLVKPGRVTAKVYAGAETTNPETTPIGEGEGYMAHTENTAYGWTAPGQSFSWGWSPAAVPWILQNVYEAYEYSGDPALLDRVYALLKEESHFYVNYMLHKAGSSSGDRLTTGVAYSPEQGPLGTDGNTYESSLVWQMLNDAIEAAKAKGDPDGLVGNTTDCSADNWAKNDSGNFTDANANRSWSCAKSLLKPIEVGDSGQIKEWYFEGALGKKKDGSTISGYQADNQHRHMSHLLGLFPGDLITIDNSEYMDAAKTSLRYRCFKGNVLQSNTGWAIGQRINSWARTGDGNTTYQLVELQLKNAMYANLFDYHAPFQIDGNFGNTSGVDEMLLQSNSTFTDTAGKKYVNYTNILPALPDAWAGGSVSGLVARGNFTVGTTWKNGKATEVRLTSNKGKQAAVKITAGGAQNYEVKNGDTAVNAKVVTNADGASLLVFDTTAGTTYTITKKASANVPVTGVTVTGANTATAGDTVTLTATVAPANATDKSVTWSTSDAAVATVNANGVVTTKKAGKVTITATSNGDKTKFGSIEITVSAATVPVTSVTVAGDAAMTVDGEQTLTATVAPATATDKTVTWKSSDATVATVDANGKVVAKKAGEVTITATAGGVSGTLKITVSDKAPTVIPVQSVTVTGKQELVEGASTTLTATVAPADATDKTVTWKSSDESVATVDKDGVVTAKKAGTVTITATAGGVSGTLHITVTAKPVETVPVTSVEVTVEAGTTVSVGKTLQATATVKPGNATNKKVTWKSSDESIATVDANGVITAKKAGKVVITATSTDGTDKSGSVEITVVDETKPTPDHKSVKADTGDVTAGKTGTVTEPKDVAGWKSRSIIKQGKLGKAEIADGTLVYAAGDKTGDDSFVVQYTMADGTVIDVTYSVTVKAAETGKNDGDGKGDGVAKTGAAVGALAGLGLMLLAVGVSVVMIRRKHSA

SEQ ID NO: 3 - DNA, 4482 bp, Bifidobacterium bifidum
atgctacacacagcatcaagaggatgctcgcgttcgtggctgcgcagactcaccgcattgatagcggtctcggcgctcgcgttcgtggcattgccgaacgtcgcggtggcggcggatccgatggaatacctcgatgtgtcgttcggcggcacgttcgctgcagacacctacaccacaggtggcgacgaggtggcgaagggccccgtgaccaagcacggcagcataccgaccaagcttgacggcggcggcatcaccctcgctggcggcaccaacggcgtgacattcacctcgaccgcgagcttcagcgagagtgggaaggtgaacaagggattccgcgccgaaatggagtaccgtacgacgcagacgcccagcaacctcgccacattgttctccgccatgggcaacatcttcgtgcgggcgaacggcagcaacctcgaatacggcttctccacgaacccttccggcagtacatggaacgactacacaaagtccgtgacgctgccttccaacaatgtgaagcacatcatccagctgacatatctgccgggagccgacggcgctgcctcgacgttgcagttgtcggtggatggcgtggccggcgagaccgccacctccgcggccggcgagctcgcggccgtcagcgattccgtcgggaacaagttcgggatcggctacgaggtgaaccccgcttccggcgcggcgagccgcggtcttgccggtgacgtgttccgcgcgcgtgtcgccgattcggacgccccgtgggagattcttgacgcatcccagctgctgcatgtcaatttcaacggcacgttcagcggcacctcatataccgcggcgagcggcgagcagatgctgggctcgctggtgtcgcgctcggccaatccgtccatctcgaactccgccgtcacgctgggcggcggcacggccggattcgatttcacgcccacggacttcaccctcggtgacaacgaggccatcacccgcccgctggtcgcggagctgcgcttcaccccgacgcagaccggcgacaaccagaccctgttcggcgcgggcggcaacctgttcctgcgctacgagtcgaacaagctcgtgttcggcgcctccaccaagtccggcgataattggaccgaccacaagatcgagtccgcggccgccacgggtgcggagcacgtcgtgtcggtggcgtacgtgcccaataaggccggcaccggcgcgaagcttgtcatgcgcgtggatggcggcgacgcccagaccaaggacatcactggtctggcttacctgaattcgagcatcaagggcaaggtcggcttcggcaacgacgtgcataccgacgcgctcagccgcggcttcgtcggctcgctgagcgagatccgcctggccgaaacctccgcgaacttcaccaccaacgaattcaagctggtctactctcaggtcagctgcgacacgtcgggcatcaaggaggcgaataccttcgacgtggagcccgccgagtgcgaggccgcgcttaagaccaagctgtccaagctgcgtccgaccgaagggcaggccgactacatcgactggggtcagatcggattcctccattacggcatcaacacgtactacaaccaggagtggggtcacggtaacgaggatccctcccgcatcaacccgaccggcctcgacaccgaccagtgggcgaagtccttcgccgacggtggcttcaagatgatcatggtgacggtcaagcaccatgacggtttcgagctgtacgactcgcggtacaacaccgagcacgactgggcaaacaccgccgtcgccaagcgcacgggggagaaggacctgttccgcaagattgtcgcctcggcgaagaaatacggcctgaaggtcggcatctactattcgccggccgattcctacatggagaggaagggcgtctggggcaacaactccgcacgcgtcgagcgcacgatccccacgctggtggagaacgacgaccgcgccggcaaggtggcttccggcaaactgcccacgttcaagtacaaggccacggattacggcgcctacatgctcaaccagctctatgagctgctgactgagtacggcgacatctccgaggtctggttcgacggtgcccaaggcaacaccgcaggcactgagcattacgactatggcgtgttctacgagatgatccgccggcttcagccccaggcaattcaggccaacgccgcatacgatgcccgatgggtgggcaacgaggacggctgggcccgtcagaccgagtggagcccgcaggcggcatacaacgacggcgtggacaaggtgtcgctcaagcctggccagatggcccccgacggtaagcttggcagcatgtcgagcgtgctgtccgagatccgcagcggcgccgccaaccagctgcactggtatccggccgaagtcgacgccaagaaccggcccggatggttctaccgtgccagccaatcgccggcgtccgtagccgaagtcgtgaagtactacgagcagtccacgggacgcaactcgcagtatctgctgaacgtcccaccgtccgataccggcaagctcgccgatgcggatgccgcgggacttaaggggctgggcgaggagctcgcccgacgctacggcaccgatcttgccctgggcaagagcgcgaccgtcgccgcgtccgcgaacgacactgcggtagcggccccgaagctgaccgacggttcgaagctctcctccgacaaggccgtgggcaatacgccgacgtacaccatcgatctgggcagcactgtcgccgtggatgcagtgaagatctccgaggacgtgcgcaatgccggccagcagatcgaaagcgccactctgcagggacgagtcaatggaacatggacgaatctggcgactatgacgacggtcgggcagcagcgcgaccttcgcttcacgtcccagaacatcgatgccatccgtctggtggtcaactcctcccgcggtccggtgcgtctgagccgtcttgaggtgttccacaccgaatccgagattcagaccggcgcccgcgcctactacatcgatccgacggcgcagaccgcgggagatggattcacgaaggacaagcccatgacgtcgatcgagcagctgcacgatgtgaccgtcgcgccaggctccgtgatcttcgtcaaggcgggcaccgagctgaccggggacttcgccgtcttcggctacggcaccaaggacgagcccatcaccgtgacgacatacggcgaaagcgacaaagccaccaccgcgagcttcgacggcatgaccgccgggctgacgctgaagcaggcgctgaaggcgctcggcaaggacgacgccggctgggtcgtggccgattccgccactgcaccggcctcccgcgtgtatgtcccgcaggatgagatcagcgtgcacgcccagtcgtcgcagaactccggcgcagaggcggcgagggcgctcgacggcgactcgtcgacgagctggcactcccagtacagcccgaccaccgcgtctgctccgcattgggtgactctcgatctcggcaaatcgcgtgagaacgtcgcctacttcgactacctcgcccgtatcgacggcaacaataacggtgccgccaaggattacgaggtgtatgtctccgacgatcccaacgattttggagcccctgtggcctcgggcacgttgaagaacgtcgcctacacgcagcgcatcaagctgacccccaagaacggacggtacgtcaagttcgtcatcaagaccgattattccggatcgaacttcggctccgcggcggaaatgaatgtcgagttgctgcccacggccgtagaggaggacaaggtcgccaccccgcagaagccgacagtggacgatgatgccgatacatacaccatccccgacatcgagggagtcgtgtacaaggtcgacggcaaggtgttggccgctggttccgtagtgaacgtgggcgatgaggacgtgaccgtcacggtcaccgccgagcccgccgacggataccgcttcccggatggtgtgacgtccccagtcacgtatgagctgacgttcaccaagaagggtggcgagaagcctccgaccgaagtcaacaaggacaagctgcacgccacgatcaccaaggctcaggcgatcgaccgttccgcctatacggacgagtcgctcaaggtgcttgatgacaagctcgccgcagcgctcaaggtctatgacgatgacaaggtgagccaggatgatgtcgatgccgccgaggcggctctgtctgcggcgatcgacgcgctgaagaccaagccgacgacccccggcggtgaaggtgagaagcctggtgaaggtgaaaagcccggtgacggcaacaagcccggtgacggcaagaagcccggcgacgtgatcgcaaagaccggcgcctccacaatgggcgttgtcttcgctgcactcgcgatggtagcgggtgcggtcgtgacgcttgaagccaagcgtaagtccaaccggtaa

SEQ ID NO: 4 - protein, 1493 aa, Bifidobacterium bifidum
MLHTASRGCSRSWLRRLTALIAVSALAFVALPNVAVAADPMEYLDVSFGGTFAADTYTTGGDEVAKGPVTKHGSIPTKLDGGGITLAGGTNGVTFTSTASFSESGKVNKGFRAEMEYRTTQTPSNLATLFSAMGNIFVRANGSNLEYGFSTNPSGSTWNDYTKSVTLPSNNVKHIIQLTYLPGADGAASTLQLSVDGVAGETATSAAGELAAVSDSVGNKFGIGYEVNPASGAASRGLAGDVFRARVADSDAPWEILDASQLLHVNFNGTFSGTSYTAASGEQMLGSLVSRSANPSISNSAVTLGGGTAGFDFTPTDFTLGDNEAITRPLVAELRFTPTQTGDNQTLFGAGGNLFLRYESNKLVFGASTKSGDNWTDHKIESAAATGAEHVVSVAYVPNKAGTGAKLVMRVDGGDAQTKDITGLAYLNSSIKGKVGFGNDVHTDALSRGFVGSLSEIRLAETSANFTTNEFKLVYSQVSCDTSGIKEANTFDVEPAECEAALKTKLSKLRPTEGQADYIDWGQIGFLHYGINTYYNQEWGHGNEDPSRINPTGLDTDQWAKSFADGGFKMIMVTVKHHDGFELYDSRYNTEHDWANTAVAKRTGEKDLFRKIVASAKKYGLKVGIYYSPADSYMERKGVWGNNSARVERTIPTLVENDDRAGKVASGKLPTFKYKATDYGAYMLNQLYELLTEYGDISEVWFDGAQGNTAGTEHYDYGVFYEMIRRLQPQAIQANAAYDARWVGNEDGWARQTEWSPQAAYNDGVDKVSLKPGQMAPDGKLGSMSSVLSEIRSGAANQLHWYPAEVDAKNRPGWFYRASQSPASVAEVVKYYEQSTGRNSQYLLNVPPSDTGKLADADAAGLKGLGEELARRYGTDLALGKSATVAASANDTAVAAPKLTDGSKLSSDKAVGNTPTYTIDLGSTVAVDAVKISEDVRNAGQQIESATLQGRVNGTWTNLATMTTVGQQRDLRFTSQNIDAIRLVVNSSRGPVRLSRLEVFHTESEIQTGARAYYIDPTAQTAGDGFTKDKPMTSIEQLHDVTVAPGSVIFVKAGTELTGDFAVFGYGTKDEPITVTTYGESDKATTASFDGMTAGLTLKQALKALGKDDAGWVVADSATAPASRVYVPQDEISVHAQSSQNSGAEAARALDGDSSTSWHSQYSPTTASAPHWVTLDLGKSRENVAYFDYLARIDGNNNGAAKDYEVYVSDDPNDFGAPVASGTLKNVAYTQRIKLTPKNGRYVKFVIKTDYSGSNFGSAAEMNVELLPTAVEEDKVATPQKPTVDDDADTYTIPDIEGVVYKVDGKVLAAGSVVNVGDEDVTVTVTAEPADGYRFPDGVTSPVTYELTFTKKGGEKPPTEVNKDKLHATITKAQAIDRSAYTDESLKVLDDKLAAALKVYDDDKVSQDDVDAAEAALSAAIDALKTKPTTPGGEGEKPGEGEKPGDGNKPGDGKKPGDVIAKTGASTMGVVFAALAMVAGAVVTLEAKRKSNR

SEQ ID NO: 5 - DNA, 2094 bp, Streptococcus pneumoniae
atgaataaaagaggtctttattcaaaactaggaatttctgttgtaggcattagtcttttaatgggagtccccactttgattcatgcgaatgaattaaactatggtcaactgtccatatctcctatttttcaaggaggttcatatcaactgaacaataagagtatagatatcagctctttgttattagataaattgtctggagagagtcagacagtagtaatgaaatttaaagcagataaaccaaactctcttcaagctttgtttggcctatctaatagtaaagcaggctttaaaaataattacttttcaattttcatgagagattctggtgagataggtgtagaaataagagacgcccaagagggaataaattatttattttctagaccagcttcattatggggaaagcataaaggacaggcagttgaaaatacactagtatttgtatctgattctaaagataaaacatacacaatgtatgttaatggaatagaagtgttctctgaaacagttgatacatttttgccaatttcaaatataaatggtatagataaggcaacactaggagctgttaatcgtgaaggtaaggaacattacctcgcaaaaggaagtattggtgaaatcagtctatttaacaaagcaattagtgatcaggaagtttcaaatattcccttgtcaaatccatttcagttaattttccaatcaggagattctactcaagctaactattttagaataccgacactatatacattaagtagtggaagagttctatcaagtattgatgcacgttatggtgggactcatgattctaaaagtaagattaatattgccacttcttatagtgatgataatgggaaaacgtggagtgagccaatttttgctatgaagtttaatgactatgaggagcagttagtttactggccacgagataataaattaaagaatagtcaaattagtggaagtgcttcattcatagattcatccattgttgaagataaaaaatctgggaaaacgatattactagctgatgttatgcctgcgggtattggaaataataatgcaaataaagccgactcaggttttaaagaaataaatggtcattattatttaaaactaaagaagaatggagataacgatttccgttatacagttagagaaaatggtgtcgtttatgatgaaacaactaataaacctacaaattatactataaatgataagtatgaagttttggagggaggaaagtctttaacagtcgaacaatattcggttgattttgatagtggctctttaagagaaaggcataatggaaaacaggttcctatgaatgttttctacaaagattcgttatttaaagtgactcctactaattatatagcaatgacaactagtcagaatagaggagagagttgggaacaatttaagttgttgcctccgttcttaggagaaaaacataatggaacttacttgtgtcctggacaaggtttagcattaaaatcaagtaacagattgatttttgcaacatatactagtggagaactaacctatctcatttcggatgatagtggtcaaacatggaagaaatcctcagcttcaattccgtttaaaaatgcaacagcagaagcacaaatggttgaactgagagatggtgtgattagaacattctttagaaccactacaggtaagatagcttatatgactagtagagattctggagaaacatggtcgaaagtttcgtatattgatggaattcaacaaacttcatatggcacacaagtatctgcaattaaatactctcaattaattgatggaaaagaagcagtcattttgagtacaccaaattctagaagtggccgtaagggaggccaattagttgtcggtttggtcaataaagaagatgatagtattgattggagataccactatgatattgatttgccttcgtatggttatgcctattctgcgattacagaattgccaaatcatcacataggtgtactgtttgaaaaatatgattcgtggtcgagaaatgaattgcatttaagcaatgtagttcagtatatagatttggaaattaatgatttaacaaaataa

SEQ ID NO: 6 - protein, 697 aa, Streptococcus pneumoniae
MNKRGLYSKLGISVVGISLLMGVPTLIHANELNYGQLSISPIFQGGSYQLNNKSIDISSLLLDKLSGESQTVVMKFKADKPNSLQALFGLSNSKAGFKNNYFSIFMRDSGEIGVEIRDAQKGINYLFSRPASLWGKHKGQAVENTLVFVSDSKDKTYTMYVNGIEVFSETVDTFLPISNINGIDKATLGAVNREGKEHYLAKGSIDEISLFNKAISDQEVSTIPLSNPFQLIFQSGDSTQANYFRIPTLYTLSSGRVLSSIDARYGGTHDSKSKINIATSYSDDNGKTWSEPIFAMKFNDYEEQLVYWPRDNKLKNSQISGSASFIDSSIVEDKKSGKTILLADVMPAGIGNNNANKADSGFKEINGHYYLKLKKNGDNDFRYTVRENGVVYNETTNKPTNYTINDKYEVLEGGKSLTVEQYSVDFDSGSLRERHNGKQVPMNVFYKDSLFKVTPTNYIAMTTSQNRGESWEQFKLLPPFLGEKHNGTYLCPGQGLALKSSNRLIFATYTSGELTYLISDDSGQTWKKSSASIPFKNATAEAQMVELRDGVIRTFFRTTTGKIAYMTSRDSGETWSKVSYIDGIQQTSYGTQVSAIKYSQLIDGKEAVILSTPNSRSGRKGGQLVVGLVNKEDDSIDWKYHYDIDLPSYGYAYSAITELPNHHIGVLFEKYDSWSRNELHLSNVVQYIDLEINDLTK

SEQ ID NO: 7 - DNA, 2076 bp, Bifidobacterium longum
atggaacatagagcgttcaagtggccgcagccacttgcgggcaacaagccccgcatctggtacggcggcgattacaaccccgaccaatggcctgaggaagtgtgggacgaagatgtagccctcatgcagcaggccggcgtcaacctcgtctccgtagccatcttctcctgggccaagcttgagcccgaagaaggcgtgtacgacttcgattggctcgaccgcgtcatcgacaagctcggcaaggccggcatcgccgtcgatctcgcctccggcaccgcatccccgccgatgtggatgacccaggcccacccggagatcctctgggtcgactaccgcggcgacgtctgccagcccggtgcccgccagcactggcgcgccaccagcccggtcttccttgactacgcgctcaacctgtgccgcaagatggccgagcactacaaggacaacccctatgtggtctcttggcatgtgagcaacgagtacggctgccacaaccgcttcgactattccgaagacgccgagcgcgccttccagaagtggtgcgagaagaagtacggcaccatcgacgctgtcaacgacgcctggggcaccgccttctgggcgcagcgcatgaacaatttctccgagatcatcccgccgcgattcatcggcgacggcaacttcatgaacccgggcaagctgcttgattggaagcgtttcagctccgacgcgctgctggacttctacaaggccgagcgcgacgccctgctcgagatcgcccccaagccgcagaccaccaacttcatggtctccgcgggctgcaccgtcctcgactacgacaagtggggtcatgacgtggacttcgtgtccaacgaccattacttctcgcccggcgaggcccacttcgacgagatggcctacgcggcctgcctcaccgacggcatcgcccgcaagaacccgtggttcctcatggaacattccacgtccgccgtcaactggcgcccgaccaactaccggctcgagcccggcgagctggtgcgcgactccctggcccatctggccatgggcgccgacgccatctgctacttccagtggcgtcagtccaaggccggcgccgagaagtggcattccgccatggtgccccacgcaggccccgactcccagatcttccgcgatgtgtgcgagctgggtgccgacctcaacaagcttgctgacgagggcctgctgagcaccaagctggtcaagtccaaggtcgccatcgtcttcgactacgagtcccagtgggccaccgagcacaccgccacccccacgcaggaggtgcgccactggaccgagccgctggactggttccgcgcgctggcggacaatggcctgaccgccgacgtggtgccggtccgcggtccttgggatgagtacgaggccgtcgtgttgccgagcctggccatcctgtccgagcagaccacgcgccgcgtgcgcgagtatgtggcgaacggcggcaagctgttcgtgacctactacaccggtctggtggacgacagggatcacgtctggctgggcggctaccccggctccattcgcgacgtggtgggcgtgcgcgtcgaggaattcgccccgatgggcaccgacgcccccggcaccatggaccaccttgacttggacaacggaaccgtggcgcacgatttcgccgacgtgatcacctccgtggccgataccgctcacgtggtcgcctccttcaaggcagataagtggaccggtttcgacggcgctcccgccatcaccgtcaacgacttcggcgacggcaaggccgcatacgtcggtgcccgtctcgggcgtgagggcttggccaagagcctgcccgcgctgctggaggaactcggcatcgagacttcggctgaggacgatcgtggtgaagtgctgcgcgtcgagcgtgcggacgaaactggcgagaaccacttcgtgttcctgttcaaccgcacccacgatgttgcggtcgtggacgtggaaggcgaaccgctggtcgcctcgctggcccaggtcaacgagtccgagcacacggccgccatccagcccaacggcgtactcgtcgtcaagctgtaa

SEQ ID NO: 8 - protein, 691 aa, Bifidobacterium longum
MEHRAFKWPQPLAGNKPRIWYGGDYNPDQWPEEVWDEDVALMQQAGVNLVSVAIFSWAKLEPEEGVYDFDWLDRVIDKLGKAGIAVDLASGTASPPMWMTQAHPEILWVDYRGDVCQPGARQHWRATSPVFLDYALNLCRKMAEHYKDNPYVVSWHVSNEYGCHNRFDYSEDAERAFQKWCEKKYGTIDAVNDAWGTAFWAQRMNNFSEIIPPRFIGDGNFMNPGKLLDWKRFSSDALLDFYKAERDALLEIAPKPQTTNFMVSAGCTVLDYDKWGHDVDFVSNDHYFSPGEAHFDEMAYAACLTDGIARKNPWFLMEHSTSAVNWRPTNYRLEPGELVRDSLAHLAMGADAICYFQWRQSKAGAEKWHSAMVPHAGPDSQIFRDVCELGADLNKLADEGLLSTKLVKSKVAIVFDYESQWATEHTATPTQEVRHWTEPLDWFRALADNGLTADVVPVRGPWDEYEAVVLPSLAILSEQTTRRVREYVANGGKLFVTYYTGLVDDRDHVWLGGYPGSIRDVVGVRVEEFAPMGTDAPGTMDHLDLDNGTVAHDFADVITSVADTAHVVASFKADKWTGFDGAPAITVNDFGDGKAAYVGARLGREGLAKSLPALLEELGIETSAEDDRGEVLRVERADETGENHFVFLFNRTHDVAVVDVEGEPLVASLAQVNESEHTAAIQPNGVLVVKL

SEQ ID NO: 9 - DNA, 1626 bp, Clostridium thermocellum
atggcagaaggggttatagtcaacggaactcagtttaaagacacatcgggaaatgtgatacatgcccatgggggaggcatgttaaagcatggtgactattattactggtacggtgaataccgggacgactccaacttgtttttgggtgtaagttgctacaggtcaaaagatcttgtaaactgggaatacagaggagaagtgctgagccgaaattccgctcctgaactgaatcactgcaatattgaaagaccgaaagtcatgtacaacgcatcaaccggtgaatttgtcatgtggatgcactgggagaacggcataaactacggtcaggcaagagcagctgttgcgtattccaaaacgcccgacggcaaattcacatacattcgaagctttcgtcccatgcaggataccggcgttatggatcatggccttccgggatatatgtcaagggactgcaatgtatttgtggacactgacggcaagggatattttatatccgcagccaatgagaacatggacctgcacctttatgagctgacacctgactataaaaatattgcatcccttaaggcaaagctgtttgtcggacagcagagggaagcaccatgccttataaagagaaacggctactattaccttattacttccggttgtacaggttggaacccgaatcaggctaaatacgcatattccaaagatttggccagtggctggtcccagctttacaatcttggtaattcaaccacctacaggtcacagccgacttttatcattcccgttcagggaagctcgggaaccagttatctttatatgggtgaccgttgggccggtgcctggggaggaaaggttaatgactcccaatatgtatggcttcccttaaacttcatatccgatacaacacttgaactgccctattatgactctgtaaagattgatgcttcttcaggaataatttccgagtacataccggacactacacgctacaagctggtaaacaaaaacagcggaaaagtcctggatgttcttgacggttctgtcgataatgcagcccagatagtccaatggaccgataacgggtctttgagtcaacagtggtaccttgtggacgtgggcggtggttataaaaagattgtaaatgtaaagagcggaagagccttggatgtaaaagacgaatccaaggaagacggtggagtattaatacaatataccagcaacggcggatataatcagcactggaaattcacagacataggtgacgggtattacaagatttccagccgccactgcggaaaacttatagatgtgcgaaaatggtcaacggaagacggcggaataattcagcagtggtccgatgccggaggaacaaatcagcattggaagctggtgcttgtatcaagtcccgagccttcaccatcaccttctccccaagtggttaaaggagatgtaaacggcgacttgaaagtaaattcaacggatttttccatgttaagaagatatttacttaaaaccattgacaattttccgacagaaaacggaaaacaggctgccgatttgaacggagacggcagaataaactcttcggatcttacaatgctgaaaagatacttgcttatggaagtggatttgtaa

SEQ ID NO: 10 - protein, 541 aa, Clostridium thermocellum
MAEGVIVNGTQFKDTSGNVIHAHGGGMLKHGDYYYWYGEYRDDSNLFLGVSCYRSKDLVNWEYRGEVLSRNSAPELNHCNIERPKVMYNASTGEFVMWMHWENGINYGQARAAVAYSKTPDGKFTYIRSFRPMQDTGVMDHGLPGYMSRDCNVFVDTDGKGYFISAANENMDLHLYELTPDYKNIASLKAKLFVGQQREAPCLIKRNGYYYLITSGCTGWNPNQAKYAYSKDLASGWSQLYNLGNSTTYRSQPTFIIPVQGSSGTSYLYMGDRWAGAWGGKVNDSQYVWLPLNFISDTTLELPYYDSVKIDASSGIISEYIPDTTRYKLVNKNSGKVLDVLDGSVDNAAQIVQWTDNGSLSQQWYLVDVGGGYKKIVNVKSGRALDVKDESKEDGGVLIQYTSNGGYNQHWKFTDIGDGYYKISSRHCGKLIDVRKWSTEDGGIIQQWSDAGGTNQHWKLVLVSSPEPSPSPSPQVVKGDVNGDLKVNSTDFSMLRRYLLKTIDNFPTENGKQAADLNGDGRINSSDLTMLKRYLLMEVDL

SEQ ID NO: 11 - DNA, 2631 bp, Paenibacillus sp.
atgaatcgacacgtcctgcttcatccgtatctccaccggaaggcgttgcctctgctcctggccttgacgctgctgacgggcatcgccctgttcccggcctccaccgcgcaggcggcgacgaccgtgacgtcgatgacgtacttctctgccaatgacggtcccgtcatctccaaatccggcgtcgggcaagccagctacggtttcgtcatgccgatcttcaacggaggcgctgcgacctggaacgatgtcgccgatgacgtcggcgttcgcgtcaaggtcggcggcagctgggtcgacattgacagcgttggcggctatgtgtacaaccagaactggggccattggaacgacagcggcacctatggctactggttcaccctctccgccacgaccgagctgcagctctactccaaggcgaacagcagcgtcacactcaactacacgctcgtcttccagaatgtcaatgaaacgaccattacctcgatgacaccgacccagggcccgcaattgaccgcagggtataccggcggcgcaggcttcacctatccggtcttcaacaacgatccctccatcccgtatgcagccgtagccggcgatctgaaggtgtacgtcaagccagtcgccagcagtacctggatcgatatcgacaacaacgcggcgagcggctggatctacgacagcaacttcggccagttcaccgaaggcggcggcggctactggttcaccgtcaccgagtcgatcaacgtcaagctcgagtccaggacgtcctcggccaacgtcgtctatacgatcaacttcccgcagccgacgcgcagcagctacacactctccgcctatgacggcacgacctacagcgccgatgcgagcggcgcgatcggtatcccgctgccgcggatcgacggcaccccggcgatcggcagcgagctcggcaacttcgtctaccagatctaccggaacggccagtgggtcgagatgagcaactcggcgcagagcagcttcgtctactcggccaatggctacaacaacatgtccgacgccaatcaatggggctactgggccgactacatctacggcctctggttccggccgatccaggaggatatgcagatccgcatcggctatccgctgaatggccagtccggcggcagcgtcggcagcaacttcgtcacctatacgctgatcggcaacccgaacgcgccgcgacccgatgtgagcgaccagggcgacgtcgagatcggcacgcccaccgatccggccatcgcaggatggaatctgtattggcaggatgaattcgccggcagcgcgctcgatctgaacaagtggaactacgagaccggctactacatcggcaacgaccccaatctgtggggctggggcaacgccgagatgcagcactatacgacgagcacgcaaaatgtcttcgtcgctgacggcaaactcaacatccgagcgctccacgattaccaatcgttcccgcaggacccgaaccgctacgcgacctactcctccggcaagatcaacaccaaggacaacatgtcgctgcagtacggccgcgtcgatatccgcgccaagctgccgactggcgatggcgtctggccggcactgtggatgctgccggaggactccgtctacggcgcatgggcggcatcaggagagatcgacatcatggaggcgaagggccgtctgcccggcacgacgagcggcgcgatccactacggcggccaatggccggtcaaccgctacctcgccggagaatgctacctcccgcaaggtacgacattcgccgacgactttaatgtgtacacgatgatctgggaagaggacaacatgaagtggtacgttaacggtgagtttttcttcaaggtgacgcgcgagcagtggtactccgtcgccgcccccaacaatccggacgcgccgttcgaccagccgttctatctgatcatgaacctggcggtcggcggccacttcgacggcgggcgtacgcccgacccgtccgacatcccggcgacgatgcagatcgactacgtgcgggtgtacaaagagggcgcgggcggcggtccgggcaacccgggcggcaacgtcgcggtgaccggcgttagcgtgaccccggcaacggcgcaggtgcaggtcggtcagaccgtctcgctgagcgccaacgtcgcgccagccaatgcaacgaacaagcaagtgacctggtcagtcgccaatggcagcatcgcctcggtgagcgccagcggcgtcgtcagtggactcgctgctggcacgacgaccgtaaccgccacgaccgcagacggcaaccgcaccgcctcggcgacgatcaccgtcgtgccgccaccgacgacgaccgtcatcatcggcgatagcgtgcgcggcatccgaaagaccggcgacaacctgctcttctacgtcaacggcgcaacctacgccgacctgcactacaaggtgaacggcggcggtcagcctaatgtcgcgatgacgcacacaggaggcggcaactacacctacccggtgcatggcctccaacaaggcgataccgtcgaatacttcttcacctacaaccccggcaacggcgcgctagacacgccttggcagacttatgtgcatggggtaacacaaggtgttgttgagtaa

SEQ ID NO: 12 - protein, 876 aa, Paenibacillus sp.
MNRHVLLHPYLHRKALPLLLALTLLTGIALFPASTAQAATTVTSMTYFSANDGPVISKSGVGQASYGFVMPIFNGGAATWNDVADDVGVRVKVGGSWVDIDSVGGYVYNQNWGHWNDSGTYGYWFTLSATTELQLYSKANSSVTLNYTLVFQNVNETTITSMTPTQGPQLTAGYTGGAGFTYPVFNNDPSIPYAAVAGDLKVYVKPVASSTWIDIDNNAASGWIYDSNFGQFTEGGGGYWFTVTESINVKLESRTSSANVVYTINFPQPTRSSYTLSAYDGTTYSADASGAIGIPLPRIDGTPAIGSELGNFVYQIYRNGQWVEMSNSAQSSFVYSANGYNNMSDANQWGYWADYIYGLWFRPIQEDMQIRIGYPLNGQSGGSVGSNFVTYTLIGNPNAPRPDVSDQGDVEIGTPTDPAIAGWNLYWQDEFAGSALDLNKWNYETGYYIGNDPNLWGWGNAEMQHYTTSTQNVFVADGKLNIRALHDYQSFPQDPNRYATYSSGKINTKDNMSLQYGRVDIRAKLPTGDGVWPALWMLPEDSVYGAWAASGEIDIMEAKGRLPGTTSGAIHYGGQWPVNRYLAGECYLPQGTTFADDFNVYTMIWEEDNMKWYVNGEFFFKVTREQWYSVAAPNNPDAPFDQPFYLIMNLAVGGHFDGGRTPDPSDIPATMQIDYVRVYKEGAGGGPGNPGGNVAVTGVSVTPATAQVQVGQTVSLSANVAPANATNKQVTWSVANGSIASVSASGVVSGLAAGTTTVTATTADGNRTASATITVVPPPTTTVIIGDSVRGIRKTGDNLLFYVNGATYADLHYKVNGGGQPNVAMTHTGGGNYTYPVHGLQQGDTVEYFFTYNPGNGALDTPWQTYVHGVTQGVVE

SEQ ID NO: 13 - DNA, 6783 bp, Artificial Sequence
  Transpsosn cassette
gccagatgattaattcctaatttttgttgacactctatcattgatagagttattttaccactccctatcagtgatagagaaaagtgaaatgaatagttcgacaaaaatctagaaataattttgtttaactttaagaaggagatatacaatttcgtcgacacacaggaaacatattaaaaattaaaacctgcaggagtttgaaggagatagaaccatggcgcagtcgaaactctatccagttgtgatggcaggtggctccggtagccgcttatggccgctttcccgcgtactttatcccaagcagtttttatgcctgaaaggcgatctcaccatgctgcaaaccaccatctgccgcctgaacggcgtggagtgcgaaagcccggtggtgatttgcaatgagcagcaccgctttattgtcgcggaacagctgcgtcaactgaacaaacttaccgagaacattattctcgaaccggcagggcgaaacacggcacctgccattgcgctggcggcgctggcggcaaaacgtcatagcccggagagcgacccgttaatgctggtattggcggcggatcatgtgattgccgatgaagacgcgttccgtgccgccgtgcgtaatgccatgccatatgccgaagcgggcaagctggtgaccttcggcattgtgccggatctaccagaaaccggttatggctatattcgtcgcggtgaagtgtctgcgggtgagcaggatatggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatatcctcgatgcctgtgaaaaagcgatgagcgccgtcgatccggatctcaattttattcgcgtggatgaagaagcgtttctcgcctgcccggaagagtcggtggattacgcggtcatggaacgtacggcagatgctgttgtggtgccgatggatgcgggctggagcgatgttggctcctggtcttcattatgggagatcagcgcccacaccgccgagggcaacgtttgccacggcgatgtgattaatcacaaaactgaaaacagctatgtgtatgctgaatctggcctggtcaccaccgtcggggtgaaagatctggtagtggtgcagaccaaagatgcggtgctgattgccgaccgtaacgcggtacaggatgtgaaaaaagtggtcgagcagatcaaagccgatggtcgccatgagcatcgggtgcatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtggttgtcgcgggaacggcaaaagtcaccattgatggtgatatcaaactgcttggtgaaaacgagtccatttatattccgctgggggcgacgcattgcctggaaaacccggggaaaattccgctcgatttaattgaagtgcgctccggctcttatctcgaagaggatgatgtggtgcgtttcgcggatcgctacggacgggtgtaaacgtcgcatcaggcaatgaatgcgaaaccgcggtgtaaataacgacaaaaataaaattggccgcttcggtcagggccaactattgcctgaaaaagggtaacgatatgaaaaaattaacctgctttaaagcctatgatattcgcgggaaattaggcgaagaactgaatgaagatatcgcctggcgcattggtcgcgcctatggcgaatttctcaaaccgaaaaccattgtgttaggcggtgatgtccgcctcaccagcgaaaccttaaaactggcgctggcgaaaggtttacaggatgcgggcgttgacgtgctggatattggtatgtccggcaccgaagagatctatttcgccacgttccatctcggcgtggatggcggcattgaagttaccgccagccataatccgatggattataacggcatgaagctggttcgcgagggggctcgcccgatcagcggagataccggactgcgcgacgtccagcgtctggctgaagccaacgactttcctcccgtcgatgaaaccaaacgcggtcgctatcagcaaatcaacctgcgtgacgcttacgttgatcacctgttcggttatatcaatgtcaaaaacctcacgccgctcaagctggtgatcaactccgggaacggcgcagcgggtccggtggtggacgccattgaagcccgctttaaagccctcggcgcgcccgtggaattaatcaaagtgcacaacacgccggacggcaatttccccaacggtattcctaacccactactgccggaatgccgcgacgacacccgcaatgcggtcatcaaacacggcgcggatatgggcattgcttttgatggcgattttgaccgctgtttcctgtttgacgaaaaagggcagtttattgagggctactacattgtcggcctgttggcagaagcattcctcgaaaaaaatcccggcgcgaagatcatccacgatccacgtctctcctggaacaccgttgatgtggtgactgccgcaggtggcacgccggtaatgtcgaaaaccggacacgcctttattaaagaacgtatgcgcaaggaagacgccatctatggtggcgaaatgagcgcccaccattacttccgtgatttcgcttactgcgacagcggcatgatcccgtggctgctggtcgccgaactggtgtgcctgaaagataaaacgctgggcgaactggtacgcgaccggatggcggcgtttccggcaagcggtgagatcaacagcaaactggcgcaacccgttgaggcgattaaccgcgtggaacagcattttagccgtgaggcgctggcggtggatcgcaccgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaaaaacgcggccgcgatatcgttgtaaaacgacggccagtgcaagaatcataaaaaatttatttgctttcaggaaaatttttctgtataatagattcataaatttgagagaggagtttttgtgagcggataacaattccccatcttagtatattagttaagtataaatacaccgcggaggacgaaggagatagaaccatgtcaaaagtcgctctcatcaccggtgtaaccggacaagacggttcttacctggcagagtttctgctggaaaaaggttacgaggtgcatggtattaagcgtcgcgcatcgtcattcaacaccgagcgcgtggatcacatttatcaggatccgcacacctgcaacccgaaattccatctgcattatggcgacctgagtgatacctctaacctgacgcgcattttgcgtgaagtacagccggatgaagtgtacaacctgggcgcaatgagccacgttgcggtctcttttgagtcaccagaatataccgctgacgtcgacgcgatgggtacgctgcgcctgctggaggcgatccgcttcctcggtctggaaaagaaaactcgtttctatcaggcttccacctctgaactgtatggtctggtgcaggaaattccgcagaaagagaccacgccgttctacccgcgatctccgtatgcggtcgccaaactgtacgcctactggatcaccgttaactaccgtgaatcctacggcatgtacgcctgtaacggaattctcttcaaccatgaatccccgcgccgcggcgaaaccttcgttacccgcaaaatcacccgcgcaatcgccaacatcgcccaggggctggagtcgtgcctgtacctcggcaatatggattccctgcgtgactggggccacgccaaagactacgtaaaaatgcagtggatgatgctgcagcaggaacagccggaagatttcgttatcgcgaccggcgttcagtactccgtgcgtcagttcgtggaaatggcggcagcacagctgggcatcaaactgcgctttgaaggcacgggcgttgaagagaagggcattgtggtttccgtcaccgggcatgacgcgccgggcgttaaaccgggtgatgtgattatcgctgttgacccgcgttacttccgtccggctgaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataagcatgagtaaacaacgagtttttattgctggtcatcgcgggatggtcggttccgccatcaggcggcagctcgaacagcgcggtgatgtggaactggtattacgcacccgcgacgagctgaacctgctggacagccgcgccgtgcatgatttctttgccagcgaacgtattgaccaggtctatctggcggcggcgaaagtgggcggcattgttgccaacaacacctatccggcggatttcatctaccagaacatgatgattgagagcaacatcattcacgccgcgcatcagaacgacgtgaacaaactgctgtttctcggatcgtcctgcatctacccgaaactggcaaaacagccgatggcagaaagcgagttgttgcagggcacgctggagccgactaacgagccttatgctattgccaaaatcgccgggatcaaactgtgcgaatcatacaaccgccagtacggacgcgattaccgctcagtcatgccgaccaacctgtacgggccacacgacaacttccacccgagtaattcgcatgtgatcccagcattgctgcgtcgcttccacgaggcgacggcacagaatgcgccggacgtggtggtatggggcagcggtacaccgatgcgcgaatttctgcacgtcgatgatatggcggcggcgagcattcatgtcatggagctggcgcatgaagtctggctggagaacacccagccgatgttgtcgcacattaacgtcggcacgggcgttgactgcactatccgcgagctggcgcaaaccatcgccaaagtggtgggttacaaaggccgggtggtttttgatgccagcaaaccggatggcacgccgcgcaaactgctggatgtgacgcgcctgcatcagcttggctggtatcacgaaatctcactggaagcggggcttgccagcacttaccagtggttccttgagaatcaagaccgctttcggggggggagctaacgcgccatttaaatcaacctcagcggtcatagctgtttcctgtgactgagcaataactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcggtatcgataagcttgatatcgaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggtcaaagtagcgatgaagccaacgctcccgttgcagggcagtttgcgcttcccctgagtgccacctttggcttaggggatcgcgtacgcaagaaatctggtgccgcttggcagggtcaagtcgtcggttggtattgcacaaaactcactcctgaaggctatgcggtcgagtccgaatcccacccaggctcagtgcaaatttatcctgtggctgcacttgaacgtgtggcctaatgaggggatcaattctctagagctcgctgatcagaagttcctattctctagaaagtataggaacttcgatggcgcctcatccctgaagccaatagggataacagggtaat

SEQ ID NO: 14 - DNA, 2851 bp, Artificial Sequence
  Integration cassette
tggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgctgggagctaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtgccgcagtctcacgcccggagcgtagcgaccgagtgagctagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcgggtaccggcagggcggggcgtaaggcgcgccatttaaatgaagttcctattccgaagttcctattctctagaaagtataggaactt

SEQ ID NO: 15 - DNA, 2858 bp, Artificial Sequence
  Integration cassette
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgggcagcattattcgtctgcagggtggtctgggtaatcagctgtttcagtttagctttggttatgccctgagcaaaattaatggtacaccgctgtatttcgacattagccattatgccgaaaacgatgatcatggtggttatcgtctgaataatctgcagattccggaagaatatctgcagtattataccccgaaaattaataatatttataaactgctggtgcgtggcagccgtctgtatccggatatttttctgtttctgggcttttgcaacgaatttcatgcctatggctacgattttgaatatattgcccagaaatggaaaagcaaaaaatacattggctactggcagagcgaacacttttttcataaacatattctggacctgaaagaattttttattccgaaaaatgtgagcgaacaggcaaatctgctggcagcaaaaattctggaaagccagagcagcctgagcattcatattcgtcgtggcgattatattaaaaacaaaaccgcaaccctgacacatggtgtttgtagcctggaatattataaaaaagccctgaacaaaatccgcgatctggcaatgattcgtgatgtgtttatctttagcgacgatatcttctggtgcaaagaaaatattgaaaccctgctgagcaaaaaatataatatttattatagcgaagatctgagccaagaagaggatctgtggctgatgagcctggcaaatcatcatattattgccaatagcagctttagttggtggggtgcatatctgggtagcagcgcaagccagattgttatttatccgaccccgtggtatgatattaccccgaaaaacacctatatcccgattgtgaaccattggatcaacgttgataaacatagcagctgctaagcggccgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacacgcgtcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctcggtaccggcagggcggggcgtaaggcgcgccatttaaatgaagttcctattccgaagttcctattctctagaaagtataggaacttcgaagcagctccag

SEQ ID NO: 16 - DNA, 2631 bp, Artificial Sequence
  Integration cassette
ggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgaagtcggcactgaccttttcccgtcgcatcaatccggtgtttctggcgttctttgtcgttgcttttctgagcggtatcgcaggcgcactgcaggctccgaccctgagtctgtttctgtccacggaagtgaaagttcgtccgctgtgggttggtctgttctataccgtcaacgcaatcgctggcattacggttagctttatcctggcgaaacgttcagattcgcgcggtgaccgtcgcaagctgattatggtgtgctatctgatggcggttggcaactgtctgctgtttgccttcaatcgtgattacctgaccctgatcacggcaggtgtgctgctggcgagcgttgccaacaccgcaatgccgcagattttcgcgctggcccgtgaatatgccgacagctctgcacgcgaagtggttatgtttagttccatcatgcgcgctcaactgagtctggcatgggtgattggtccgccgctgtcctttatgctggcgctgaattacggttttaccctgatgttctcaatcgcggccggcattttcgttctgtcggccctggtcgtgtggtttatcctgccgagtgtcccgcgtgcagaaccggttgtcgatgcaccggtggttgtccagggttcactgttcgcagacaaaaacgttctgctgctgtttatcgcgtcgatgctgatgtggacctgcaatacgatgtatattatcgatatgccgctgtacattaccgcaagcctgggtctgccggaacgtctggctggtctgctgatgggtaccgcagctggcctggaaattccgatcatgctgctggcgggttattctgtgcgttactttggcaaacgcaagattatgctgttcgctgttctggcgggtgtcctgttttataccggcctggttctgtttaaattcaagacggccctgatgctgctgcagatctttaacgcaattttcatcggtattgtggctggcattggtatgctgtacttccaagatctgatgccgggtcgtgcaggtgcagcaaccacgctgtttaccaatagcatctctacgggtgtcattctggcaggcgtgctgcaaggcggtctgaccgaaacgtggggccatgacagcgtctatgtgatggcgatggtcctgtctattctggccctgattatctgtgcacgtgtgcgcgaagcttaaatcgatactagcataaccccttggggcctctaaacgcgtcgacacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcatgtatgaatatcctccttagttcctattccgaagttcctattctctagaaagtataggaacttcggcgcgtcctacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaaggcgcgccatttaaatgaagttcctattccgaagttcctattctctagaaagtataggaacttc

SEQ ID NO: 17 - DNA, 4259 bp, Artificial Sequence
  Integration cassette
ttactcagcaataaactgatattccgtcaggctggaatactcttcgccaggacgcaggaagcagtccggttgcggccattcagggtggttcgggctgtccggtagaaactcgctttccagagccagcccttgccagtcggcgtaaggttcggttccccgcgacggtgtgccgccgaggaagttgccggagtagaattgcagagccggagcggtggtgtagaccttcagctgcaatttttcatctgctgaccagacatgcgccgccactttcttgccatcgcctttggcctgtaacaagaatgcgtgatcgtaacctttcactttgcgctgatcgtcgtcggcaagaaactcactggcgatgattttggcgctgcggaaatcaaaagacgttccggcgacagatttcaggccgtcgtgcggaatgccgccttcatcaaccggcagatattcgtccgccagaatctgcaacttgtgattgcgcacgtcagactgctcgccgtcaagattgaaatagacgtgattagtcatattcaccgggcaaggtttatcaactgtggcgcgataagtaatggagatacggttatcgtcggtcagacgatattgcaccgtcgcgccgagattacccgggaagccctgatcaccatcatctgaactcagggcaaacagcacctgacgatcgttctggttcacaatctgccagcgacgtttgtcgaacccttccggcccgccgtgcagctggttaacgccctgacttggcgaaagcgtcacggtttcaccgtcaaaggtataacggctattggcgatacggttggcataacgaccaatagaggcccccagaaacgcggcctgatcctgatagcattccgggctggcacagccgagcagcgcctcgcggacgctgccatcggaaagcggaatacgggcggaaagtaaagtcgcaccccagtccatcagcgtgactaccatccctgcgttgttacgcaaagttaacagtcggtacggctgaccatcgggtgccagtgcgggagtttcgttcagcactgtcctgctccttgtgatggtttacaaacgtaaaaagtctctttaatacctgtttttgcttcatattgttcagcgacagcttgctgtacggcaggcaccagctcttccgggatcagcgcgacgatacagccgccaaatccgccgccggtcatgcgtacgccacctttgtcgccaatcacagctttgacgatttctaccagagtgtcaatttgcggcacggtgatttcgaaatcatcgcgcatagaggcatgagactccgccatcaactcgcccatacgtttcaggtcgccttgctccagcgcgctggcagcttcaacggtgcgggcgttttcagtcagtatatgacgcacgcgttttgccacgatcgggtccagttcatgcgcaacagcgttgaactcttcaatggtgacatcacgcagggctggctgctggaagaaacgcgcaccggtttcgcactgttcacgacgggtgttgtattcgctgccaaccagggtacgtttgaagttactgttgatgatgacgacagccacacctttgggcatggaaactgctttggtccccagtgagcggcaatcgatcagcaaggcatgatctttcttgccgagcgcggaaattagctgatccatgatcccgcagttacagcctacaaactggttttctgcttcctgaccgttaagcgcgatttgtgcgccgtccagcggcagatgataaagctgctgcaatacggttccgaccgcgacttccagtgaagcggaagaacttaacccggcaccctgcggcacattgccgctgatcaccatgtccacgccgccgaagctgttgttacgcagttgcagatgtttcaccacgccacgaacgtagttagcccattgatagttttcatgtgcgacaatgggcgcatcgagggaaaactcgtcgagctgattttcataatcggctgccatcacgcgaactttacggtcatcgcgtggtgcacaactgatcacggtttgataatcaatcgcgcagggcagaacgaaaccgtcgttgtagtcggtgtgttcaccaatcaaattcacgcggccaggcgcctgaatggtgtgagtggcagggtagccaaatgcgttggcaaacagagattgtgttttttctttcagactcatttcttacactccggattcgcgaaaatggatatcgctgactgcgcgcaaacgctctgctgcctgttctgcggtcaggtctcgctgggtctctgccagcatttcataaccaaccataaatttacgtacggtggcggagcgcagcagaggcggataaaagtgcgcgtgcagctgccagtgttgattctcttcgccattaaatggcgcgccgtgccagcccatagagtaggggaaggagcactggaagaggttgtcataacgactggtcagctttttcaacgccagcgccagatcgctgcgctgggcgtcggtcaaatcggtgatccgtaaaacgtgggctttgggcagcagtagcgtttcgaacggccaggcagcccagtaaggcacgacggctaaccagtgttcggtttcgacaacggtacggctaccgtctgccagctcgcgctgaacataatccaccagcattggtgatttctgttcggcaaaatattctttttgcaggcggtcttcgcgctcagcttcgttaggcaggaagctatttgcccaaatctgaccgtgcggatgcgggttagagcagcccatcgccgcgcctttgttttcaaaaacctgcacccatgggtacgttttccccagttctgcggtttgctcctgccaggttttgacgatttccgtcaatgctgcaacgctgagctctggcagcgttttactgtgatccggtgaaaagcagatcacccggctggtgccgcgcgcgctctggcaacgcatcagcggatcgtgactttctggcgcatctggcgtgtcagacatcaaagccgcaaagtcattagtgaaaacgtaagtcccggtgtaatcggggtttttatcgcctgtcacccgcacattacctgcgcagaggaagcaatctggatcgtgcgcaggtaacacctgtttggctggcgtttcctgcgccccctgccaggggcgcttagcgcggtgcggtgaaaccagaatccattgcccggtgagcgggttgtagcggcgatgtggatgatcaacgggattaaattgcgtcatggtcgttccttaatcgggatatccctgtggatggcgtgactgccagtgccaggtgtcctgcgccatttcatcgagtgtgcgcgttacgcgccagttcagttcacggtcggctttgctggcgtccgcccagtaggccggaaggtcgccctcgcgacgcggtgcaaaatgataattaaccggtttgccgcaggctttgctgaaggcattaaccacgtccagcacgctgttgcctacgccagcgccgaggttgtagatgtgtacgcctggcttgttcgccagtttttccatcgccacgacgtgaccgtccgccagatccattacgtggatgtaatcgcgtacgccagtaccatcttcggtcggataatcgttaccaaaaatcgccagcgagtcgcgacggcctacagcaacctgggcgatgtatggcatcaggttattcggaatgccttgcggatcttcgcccatatcgcccgacggatgcgcgccaaccgggttgaagtagcgcagcagggcaatgctccagtccggctgggctttttgcagatcggtgaggatctgttccaccatcagcttgcttttgccgtaagggctttgcggtgtgccggtcgggaagctttcaacgtatggaattttgggctgatcgccataaacggtggcggaggagctaaaaataaagtttttgacgttagcggcgcgcatggcgctaatcaggcgcagagtgccgttgacattgttgtcgtaatattccagcggtttttgtaccgattcgcccacggctttcagcccggcgaagtggatcacggtgtcgatagcgtgatcgtgcaggatctcggtcatcaacgcttcgttacgaatatcgccttcaacaaacgttggatgtttgccgcctaaacgctcgataacaggcagtacgctgcgcttactgttacagaggttatcaagaatgatgacatcatgaccgttttgcagtaattgcacacaggtatgacttccaatgtaaccgctaccaccggtaaccagaactctcat

SEQ ID NO: 18 - DNA, 4223 bp, Artificial Sequence
  Integration cassette
tggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgcaaaaactactatctttaccgtccaatctggttcagtcttttcatgaactggagagggtgaatcgtaccgattggttttgtacttccgacccggtaggtaagaaacttggttccggtggtggaacatcctggctgcttgaagaatgttataatgaatattcagatggtgctacttttggagagtggcttgaaaaagaaaaaagaattcttcttcatgcgggtgggcaaagccgtcgtttacccggctatgcaccttctggaaagattctcactccggttcctgtgttccggtgggagagagggcaacatctgggacaaaatctgctttctctgcaacttcccctatatgaaaaaatcatgtctttggctccggataaactccatacactgattgcgagtggtgatgtctatattcgttcggagaaacctttgcagagtattcccgaagcggatgtggtttgttatggactgtgggtagatccgtctctggctacccatcatggcgtgtttgcttccgatcgcaaacatcccgaacaactcgactttatgcttcagaagccttcgttggcagaattggaatctttatcgaagacccatttgttcctgatggacatcggtatatggcttttgagtgaccgtgccgtagaaatcttgatgaaacgttctcataaagaaagctctgaagaactaaagtattatgatctttattccgattttggattagctttgggaactcatccccgtattgaagacgaagaggtcaatacgctatccgttgctattctgcctttgccgggaggagagttctatcattacgggaccagtaaagaactgatttcttcaactctttccgtacagaataaggtttacgatcagcgtcgtatcatgcaccgtaaagtaaagcccaatccggctatgtttgtccaaaatgctgtcgtgcggatacctctttgtgccgagaatgctgatttatggatcgagaacagtcatatcggaccaaagtggaagattgcttcacgacatattattaccggggttccggaaaatgactggtcattggctgtgcctgccggagtgtgtgtagatgtggttccgatgggtgataagggctttgttgcccgtccatacggtctggacgatgttttcaaaggagatttgagagattccaaaacaaccctgacgggtattccttttggtgaatggatgtccaaacgcggtttgtcatatacagatttgaaaggacgtacggacgatttacaggcagtttccgtattccctatggttaattctgtagaagagttgggattggtgttgaggtggatgttgtccgaacccgaactggaggaaggaaagaatatctggttacgttccgaacatttttctgcggacgaaatttcggcaggtgccaatctgaagcgtttgtatgcacaacgtgaagagttcagaaaaggaaactggaaagcattggccgttaatcatgaaaaaagtgttttttatcaacttgatttggccgatgcagctgaagattttgtacgtcttggtttggatatgcctgaattattgcctgaggatgctctgcagatgtcacgcatccataaccggatgttgcgtgcgcgtattttgaaattagacgggaaagattatcgtccggaagaacaggctgcttttgatttgcttcgtgacggcttgctggacgggatcagtaatcgtaagagtaccccaaaattggatgtatattccgatcagattgtttggggacgtagccccgtgcgcatcgatatggcaggtggatggaccgatactcctccttattcactttattcgggaggaaatgtggtgaatctagccattgagttgaacggacaacctcccttacaggtctatgtgaagccgtgtaaagacttccatatcgtcctgcgttctatcgatatgggtgctatggaaatagtatctacgtttgatgaattgcaagattataagaagatcggttcacctttctctattccgaaagccgctctgtcattggcaggctttgcacctgcgttttctgctgtatcttatgcttcattagaggaacagcttaaagatttcggtgcaggtattgaagtgactttattggctgctattcctgccggttccggtttgggcaccagttccattctggcttctaccgtacttggtgccattaacgatttctgtggtttagcctgggataaaaatgagatttgtcaacgtactcttgttcttgaacaattgctgactaccggaggtggatggcaggatcagtatggaggtgtgttgcagggtgtgaagcttcttcagaccgaggccggctttgctcaaagtccattggtgcgttggctacccgatcatttatttacgcatcctgaatacaaagactgtcacttgctttattataccggtataactcgtacggcaaaagggatcttggcagaaatagtcagttccatgttcctcaattcatcgttgcatctcaatttactttcggaaatgaaggcgcatgcattggatatgaatgaagctatacagcgtggaagttttgttgagtttggccgtttggtaggaaaaacctgggaacaaaacaaagcattggatagcggaacaaatcctccggctgtggaggcaattatcgatctgataaaagattataccttgggatataaattgccgggagccggtggtggcgggtacttatatatggtagcgaaagatccgcaagctgctgttcgtattcgtaagatactgacagaaaacgctccgaatccgcgggcacgttttgtcgaaatgacgttatctgataagggattccaagtatcacgatcataactgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaagacggccagtgaattcgagctcggtacctaccgttcgtataatgtatgctatacgaagttatcgagctctagagaatgatcccctcattaggccacacgttcaagtgcagcgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcgtagaatttcgacgacctgcagccaagcataacttcgtataatgtatgctatacgaacggtaggatcctctagagtcgacctgcaggcatgagatgtgtataagagacag

SEQ ID NO: 19 - DNA, 3792 bp, Artificial Sequence
  Integration cassette
gggaattgattctggtaccaaatgagtcgaccggccagatgattaattcctaatttttgttgacactctatcattgatagagttattttaccactccctatcagtgatagagaaaagtgaaatgaatagttcgacaaaaatctagaaataattttgtttaactttaagaaggagatatacaaatgattacccgcaaaaggcgggccaggacaatccatagccgatatccaatcggaatttacgggagcatagtaatgacagatattgcacagttgcttggcaaagacgccgacaaccttttacagcaccgttgtatgactattccttctgaccagctttatctccccggacatgactacgtagaccgcgtgatgattgacaataatcgcccgccagcggtgttacgtaatatgcagacgttgtacaacactgggcgtctggctggcacaggatatctttctattctgccggttgaccagggcgttgagcactctgccggagcttcatttgctgctaacccgctctactttgacccgaaaaacattgttgaactggcgatcgaagcgggctgtaactgtgtggcatcaacttacggcgtgttggcgtcggtatcgcggcgctatgcgcatcgcattccattcctcgtcaaacttaatcacaacgagacgctaagttacccgaacacctacgatcaaacgctgtatgccagcgtggagcaggccttcaacatgggcgcggtggcggttggtgcgactatctattttggttcggaagagtcacgtcgccagattgaagaaatttctgcggcttttgaacgtgcgcacgagctgggcatggtgacagtgctgtgggcctatttgcgtaactccgcctttaagaaagatggcgttgattaccatgtttccgccgacctgaccggtcaggcaaaccatctggcggcgaccataggtgcagatatcgtcaaacaaaaaatggcggaaaataacggcggctataaagcaattaattacggttataccgacgatcgcgtgtacagcaagttaaccagcgaaaacccgattgatctggtgcgttatcagttagctaactgctatatgggccgggccgggttgataaactccggcggtgctgcaggcggtgaaactgacctcagcgatgcagtgcgtactgcggttatcaacaaacgcgctggcggaatggggctgattcttggacgtaaggcgttcaagaaatcgatggctgacggcgtgaaactgattaacgccgtgcaggatgtttatctcgatagcaaaattactatcgcctaagaggatcgagatctcgatcccgcgaaattaatacgactcactataggggaattgtgagcggataacaattcccctctagaaataattttgtttaactttaagaaggagatataccatgggccatcatcatcatcatcatcatcatcatcacagcagcggccatatcgaaggtcgtcatatggcggtgaaagaagcgaccagcgagaccaagaagcgtagcggttacgagatcattaccctgaccagctggctgctgcaacaagaacagaagggtatcattgacgcggaactgaccatcgttctgagcagcattagcatggcgtgcaaacagatcgcgagcctggtgcaacgtgcgaacattagcaacctgaccggtacccaaggcgcggttaacatccagggtgaagaccaaaagaaactggatgttattagcaacgaggtgttcagcaactgcctgcgtagcagcggtcgtaccggcatcattgcgagcgaggaagaggacgtggcggttgcggtggaagagagctacagcggtaactatatcgtggtttttgacccgctggatggcagcagcaacctggatgcggctgtgagcaccggtagcatcttcggcatttacagcccgaacgacgagagcctgccggattttggtgacgatagcgacgataacaccctgggcaccgaagagcaacgttgcatcgttaacgtgtgccaaccgggtagcaacctgctggcggcgggctactgcatgtatagcagcagcgttgcgttcgtgctgaccattggcaagggcgttttcgtgtttaccctggacccgctgtacggtgaattcgtgctgacccaggagaacctgcaaatcccgaagagcggtgaaatttacagctttaacgagggcaactataaactgtgggatgaaaacctgaagaaatatatcgacgatctgaaggaaccgggtccgagcggtaaaccgtacagcgcgcgttatatcggtagcctggttggcgacttccaccgtaccctgctgtacggtggcatttacggttatccgcgtgataagaaaagcaagaacggcaaactgcgtctgctgtatgaatgcgcgccgatgagctttattgttgagcaggcgggtggcaaaggtagcgacggccaccagcgtgtgctggatatccaaccgaccgaaattcaccagcgtgttccgctgtacattggtagcaccgaagaggttgaaaaagttgaaaagtatctggcgtaatcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtggacggccagtgaattcgagctcggtacctaccgttcgtataatgtatgctatacgaagttatcgagctctagagaatgatcccctcattaggccacacgttcaagtgcagcgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcgtagaatttcgacgacctgcagccaagcataacttcgtataatgtatgctatacgaacggtaggatcctctagagtcgacctgcaggc

SEQ ID NO: 20 - DNA, 5917 bp, Artificial Sequence
  Transposon cassette
acaggttggctgataagtccccggtctagcttgcatgcagattgcagcattacacgtcttgatttgacggctagctcagtcctaggtacagtgctagcactgctttgtggaaggagatagacttatggcggatccgatggaatacctcgatgtgtcgttcggcggcacgttcgctgcagacacctacaccacaggtggcgacgaggtggcgaagggccccgtgaccaagcacggcagcataccgaccaagcttgacggcggcggcatcaccctcgctggcggcaccaacggcgtgacattcacctcgaccgcgagcttcagcgagagtgggaaggtgaacaagggattccgcgccgaaatggagtaccgtacgacgcagacgcccagcaacctcgccacattgttctccgccatgggcaacatcttcgtgcgggcgaacggcagcaacctcgaatacggcttctccacgaacccttccggcagtacatggaacgactacacaaagtccgtgacgctgccttccaacaatgtgaagcacatcatccagctgacatatctgccgggagccgacggcgctgcctcgacgttgcagttgtcggtggatggcgtggccggcgagaccgccacctccgcggccggcgagctcgcggccgtcagcgattccgtcgggaacaagttcgggatcggctacgaggtgaaccccgcttccggcgcggcgagccgcggtcttgccggtgacgtgttccgcgcgcgtgtcgccgattcggacgccccgtgggagattcttgacgcatcccagctgctgcatgtcaatttcaacggcacgttcagcggcacctcatataccgcggcgagcggcgagcagatgctgggctcgctggtgtcgcgctcggccaatccgtccatctcgaactccgccgtcacgctgggcggcggcacggccggattcgatttcacgcccacggacttcaccctcggtgacaacgaggccatcacccgcccgctggtcgcggagctgcgcttcaccccgacgcagaccggcgacaaccagaccctgttcggcgcgggcggcaacctgttcctgcgctacgagtcgaacaagctcgtgttcggcgcctccaccaagtccggcgataattggaccgaccacaagatcgagtccgcggccgccacgggtgcggagcacgtcgtgtcggtggcgtacgtgcccaataaggccggcaccggcgcgaagcttgtcatgcgcgtggatggcggcgacgcccagaccaaggacatcactggtctggcttacctgaattcgagcatcaagggcaaggtcggcttcggcaacgacgtgcataccgacgcgctcagccgcggcttcgtcggctcgctgagcgagatccgcctggccgaaacctccgcgaacttcaccaccaacgaattcaagctggtctactctcaggtcagctgcgacacgtcgggcatcaaggaggcgaataccttcgacgtggagcccgccgagtgcgaggccgcgcttaagaccaagctgtccaagctgcgtccgaccgaagggcaggccgactacatcgactggggtcagatcggattcctccattacggcatcaacacgtactacaaccaggagtggggtcacggtaacgaggatccctcccgcatcaacccgaccggcctcgacaccgaccagtgggcgaagtccttcgccgacggtggcttcaagatgatcatggtgacggtcaagcaccatgacggtttcgagctgtacgactcgcggtacaacaccgagcacgactgggcaaacaccgccgtcgccaagcgcacgggggagaaggacctgttccgcaagattgtcgcctcggcgaagaaatacggcctgaaggtcggcatctactattcgccggccgattcctacatggagaggaagggcgtctggggcaacaactccgcacgcgtcgagcgcacgatccccacgctggtggagaacgacgaccgcgccggcaaggtggcttccggcaaactgcccacgttcaagtacaaggccacggattacggcgcctacatgctcaaccagctctatgagctgctgactgagtacggcgacatctccgaggtctggttcgacggtgcccaaggcaacaccgcaggcactgagcattacgactatggcgtgttctacgagatgatccgccggcttcagccccaggcaattcaggccaacgccgcatacgatgcccgatgggtgggcaacgaggacggctgggcccgtcagaccgagtggagcccgcaggcggcatacaacgacggcgtggacaaggtgtcgctcaagcctggccagatggcccccgacggtaagcttggcagcatgtcgagcgtgctgtccgagatccgcagcggcgccgccaaccagctgcactggtatccggccgaagtcgacgccaagaaccggcccggatggttctaccgtgccagccaatcgccggcgtccgtagccgaagtcgtgaagtactacgagcagtccacgggacgcaactcgcagtatctgctgaacgtcccaccgtccgataccggcaagctcgccgatgcggatgccgcgggacttaaggggctgggcgaggagctcgcccgacgctacggcaccgatcttgccctgggcaagagcgcgaccgtcgccgcgtccgcgaacgacactgcggtagcggccccgaagctgaccgacggttcgaagctctcctccgacaaggccgtgggcaatacgccgacgtacaccatcgatctgggcagcactgtcgccgtggatgcagtgaagatctccgaggacgtgcgcaatgccggccagcagatcgaaagcgccactctgcagggacgagtcaatggaacatggacgaatctggcgactatgacgacggtcgggcagcagcgcgaccttcgcttcacgtcccagaacatcgatgccatccgtctggtggtcaactcctcccgcggtccggtgcgtctgagccgtcttgaggtgttccacaccgaatccgagattcagaccggcgcccgcgcctactacatcgatccgacggcgcagaccgcgggagatggattcacgaaggacaagcccatgacgtcgatcgagcagctgcacgatgtgaccgtcgcgccaggctccgtgatcttcgtcaaggcgggcaccgagctgaccggggacttcgccgtcttcggctacggcaccaaggacgagcccatcaccgtgacgacatacggcgaaagcgacaaagccaccaccgcgagcttcgacggcatgaccgccgggctgacgctgaagcaggcgctgaaggcgctcggcaaggacgacgccggctgggtcgtggccgattccgccactgcaccggcctcccgcgtgtatgtcccgcaggatgagatcagcgtgcacgcccagtcgtcgcagaactccggcgcagaggcggcgagggcgctcgacggcgactcgtcgacgagctggcactcccagtacagcccgaccaccgcgtctgctccgcattgggtgactctcgatctcggcaaatcgcgtgagaacgtcgcctacttcgactacctcgcccgtatcgacggcaacaataacggtgccgccaaggattacgaggtgtatgtctccgacgatcccaacgattttggagcccctgtggcctcgggcacgttgaagaacgtcgcctacacgcagcgcatcaagctgacccccaagaacggacggtacgtcaagttcgtcatcaagaccgattattccggatcgaacttcggctccgcggcggaaatgaatgtcgagttgctgcccacggccgtagaggaggacaaggtcgccaccccgcagaagccgacagtggacgatgatgccgatacatacaccatccccgacatcgagggagtcgtgtacaaggtcgacggcaaggtgttggccgctggttccgtagtgaacgtgggcgatgaggacgtgaccgtcacggtcaccgccgagcccgccgacggataccgcttcccggatggtgtgacgtccccagtcacgtatgagctgacgttcaccaagaagggtggcgagaagcctccgaccgaagtcaacaaggacaagctgcacgccacgatcaccaaggctcaggcgatcgaccgttccgcctatacggacgagtcgctcaaggtgcttgatgacaagctcgccgcagcgctcaaggtctatgacgatgacaaggtgagccaggatgatgtcgatgccgccgaggcggctctgtctgcggcgatcgacgcgctgaagaccaagccgacgacccccggcggtgaaggtgagaagcctggtgaaggtgaaaagcccggtgacggcaacaagcccggtgacggcaagaagcccggcgacgtgatcgcaaagaccggcgcctccacaatgtaactagcataaccccttggggcctctaaacgggtcttgaggggttttttgctgaaaccaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgggatccaggccggcctgttaacgaattaatcttccgcggcggtatcgataagcttgatatcgaattccgaagttcctattctctagacgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctaccgttcgtataatgtatgctatacgaagttatcgagctctagagaatgatcccctcattaggccacacgttcaagtgcagcgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccagatttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcgtagaatttcgacgacctgcagccaagcataacttcgtataatgtatgctatacgaacggtaggatcctctagagtcgaccaggtggcacttttcgggcagaccggggacttatcagccaacctgt

(19) United States
(12) Patent Application Publication
(10) Pub. No.: US 2021/0363557 A1
(43) Pub. Date: Nov. 25, 2021

(54) USE OF GLYCOSIDASES IN THE PRODUCTION OF OLIGOSACCHARIDES

(71) Applicant: Jennewein Biotechnologie GmbH, Rheinbreitbach (DE)

(72) Inventors: Stefan JENNEWEIN, Bad Honnef (DE); Dirk WARTENBERG, Gau-Algesheim (DE)

(21) Appl. No.: 17/054,950

(22) PCT Filed: May 13, 2019
(86) PCT No.: PCT/EP2019/062160

§ 371 Date: Nov. 12, 2020

(30) Foreign Application Priority Data
May 16, 2018 (EP) 18172658.9

(51) Int. Cl. C12P 19/00; C12N 1/20; C12N 9/10; C12N 9/24; A23L 33/00

(52) U.S. Cl. CPC C12P 19/00; C12N 1/20; A23L 33/40; C12N 9/2405; C12N 9/1051

(57) ABSTRACT

Disclosed is a method for the production of a desired oligosaccharide using a genetically-engineered microbial host cell, and said genetically-engineered microbial host cell which has been genetically engineered to express a heterologous glycosidase which is able to intracellularly degrade metabolic saccharide by-products that are generated during the intracellular biosynthesis of the desired oligosaccharide.

The present invention relates to the production of oligosaccharides by microbial fermentation. More specifically, the present invention concerns the use of glycosidases to improve the production of desired oligosaccharides by microbial fermentation.

### BACKGROUND

Human milk contains a unique mixture of different oligosaccharides called Human Milk Oligosaccharides (HMOs). More than 150 structurally different oligosaccharides were identified in human milk thus far. With very few exceptions, the HMOs are characterized by a lactose moiety at their reducing end, and many HMOs contain a fucose residue and/or an N-acetylneuraminic acid residue at their non-reducing end. Generally, the monosaccharide residues of HMOs are derived from D-glucose, D-galactose, N-acetylglucosamine, L-fucose and N-acetylneuraminic acid. The importance of HMOs for infant nutrition is directly linked to their unique biological activities including protection of the neonate from pathogens, supporting development of the infant's immune system and cognitive abilities. Therefore, there is a strong interest in preparing HMOs in a commercial scale.

Besides chemical synthesis of individual HMOs, considerable progress has been made in the development of producing HMOs by microbial fermentation using genetically-modified microorganisms which overexpress a heterologous glycosyltransferase. Upon cultivation of such microorganisms in a medium and under conditions permissive for the microorganism to express said heterologous glycosyltransferase, a HMO can be produced by said microorganism and recovered from the culture medium or cell lysate.

However, glycosyltransferases often possess enzymatic side activities such that their overexpression for producing a desired oligosaccharide typically leads to by-products which are undesired. Typically, these by-products are oligosaccharides too, but have to be removed from the preparation of the desired oligosaccharide for the product's commercial use. However, removing such by-products from the desired oligosaccharide is difficult and cumbersome. One approach of removing such by-products involves the use of glycosidases that are either exogenously added to a reaction mixture/cell medium containing desired and undesired oligosaccharides or produced by a genetically engineered microorganism upon induction at a specific point of time at the end of the fermentation process for producing the desired oligosaccharide.

International Publication No. WO 2015/032412 A1 concerns the use of fucose and discloses a method wherein a genetically-modified cell expressing a heterologous fucosyltransferase is cultivated in the presence of lactose to produce and secrete a mixture of 2′-fucosyllactose (2′-FL) and difucosyllactose (DFL) into an extracellular space of the culture medium in high yield. The saccharides are separated and subjected to hydrolysis by an acid or by a fucosidase to produce fucose in high yields.

International Publication No. WO 2104/090261 A1 discloses a method to form a mixture containing at least one of 2′-FL and 3-fucosyllactose (3-FL), wherein DFL is subjected to partial hydrolysis, e.g. enzymatic hydrolysis or acid hydrolysis. In the enzymatic hydrolysis, DFL is exposed to a fucosidase that can release one of the fucose residues from DFL. DFL (10 mM) was incubated with the 1,2-α-L-fucosidase from Xanthomonas manihotis at 37° C. in an incubation buffer, and hydrolysis of DFL was followed by HPLC. After 18 hours, DFL was partially hydrolyzed to 3-FL and fucose. No lactose was detected.

European Patent Application No. EP 2 845 905 A1 concerns the production of oligosaccharides and discloses the use of one or more glycosidases in the process for the production and/or purification of an oligosaccharide. The process comprises a) cultivating a host microorganism suitable for the production of a desired oligosaccharide under conditions and in a medium permissive for the production of said desired oligosaccharide, whereby the oligosaccharide and, where applicable, biosynthetic saccharide intermediates and/or side products are produced; b) using a glycosidase in the medium the host microorganism is cultivated in, in order to degrade biosynthetic saccharide intermediates and/or saccharide side products and/or unused saccharide substrates; and c) recovering the desired oligosaccharide. In an embodiment, said glycosidase is endogenously produced in the host microorganism, wherein the glycosidase is a glycosidase that is not naturally occurring in the host microorganism, and wherein the expression of said glycosidase in said host microorganism is inducible such that the expression can be initiated after a sufficient and/or essentially maximum amount of desired oligosaccharide has been produced during cultivation of the host microorganism.

In summary, prior art discloses the use of glycosidases to remove undesired oligosaccharides from a mixture of desired and undesired oligosaccharides by hydrolysis of the undesired oligosaccharides in a reaction mixture/cell medium. However, these approaches comprise biosynthesis of the undesired oligosaccharides by the microorganism including the use of substrates and energy, and these approaches require removal of the degradation products of the undesired oligosaccharides from the desired oligosaccharide.

It was therefore an object of the present invention to provide a method for the production of a desired oligosaccharide by microbial fermentation without concomitant production/accumulation of undesired saccharide by-products, i.e. undesired oligosaccharides, in the cell medium containing the microorganism to be fermented.

The object is solved by providing a genetically-engineered microbial host cell being able to produce a desired oligosaccharide, wherein said microbial host cell expresses a heterologous glycosidase which is able to degrade metabolic by-products intracellularly that are generated during the intracellular biosynthesis of the desired oligosaccharide, thus preventing the formation of a mixture of desired and undesired saccharides in the culture medium. Said degradation products may then be utilized by the microbial host cell's metabolism, for example for the biosynthesis of the desired oligosaccharide.

Table 1 provides a comprehensive overview of desired oligosaccharides and conceivable precursors that are added for and/or undesired saccharide by-products that are generated during the production of the desired oligosaccharide.

TABLE 1
Overview of desired oligosaccharides and conceivable precursors that
are added for and/or undesired saccharide by-products that are generated
during the production of the desired oligosaccharide.

| | Conceivable precursors added to and/or by- |
|---|---|
| | products generated during production of the |
| Desired oligosaccharides | desired oligosaccharides |
| fucosylated | L-fucose |
| trisaccharides: | glucose |
| 2′-fucosyllactose (2′-FL) | galactose |
| or | lactose |
| 3-fucosyllactose (3-FL) | fucosylated galactose |
| | fucosylated glucose |
| | 3-fucosyllactose |
| | 2′-fucosyllactose |
| | difucosyllactose |
| sialylated trisaccharides: | N-acetylglucosamine |
| 3′-sialyllactose (3′-SL) | N-acetylmannosamine |
| or | N-acetylneuraminic acid |
| 6′-sialyllactose (6′-SL) | glucose |
| | galactose |
| | lactose |
| | sialylated galactose |
| | sialylated glucose |
| | sialylated N-acetylglucosamine |
| | sialylated N-acetylmannosamine |
| | 6′-sialyllactose |
| | 3′-sialyllactose |
| | 3-sialyllactose |
| | 6-sialyllactose |
| | disialyllactose |
| | KDO-lactose |
| N-acetylglucosaminylated | glucose |
| trisaccharides: | galactose |
| lacto-N-triose II (LNT-II) | lactose |
| | N-acetylglucosaminylated galactose |
| | galactosylated lactose |
| | glucosylated lactose |
| galactosylated | glucose |
| tetrasaccharides: | galactose |
| lacto-N-tetraose (LNT) | N-acetylglucosamine |
| or | lactose |
| lacto-N-neotetraose (LNnT) | glucosylated galactose |
| | galactosylated galactose |
| | N-acetylglucosaminylated galactose |
| | galactosylated lactose |
| | glucosylated lactose |
| | lacto-N-triose II |
| | lacto-N-neotetraose |
| | lacto-N-tetraose |
| | galactosylated lacto-N-tetraose |
| | glucosylated lacto-N-tetraose |
| | N-acetylglucosaminylated lacto-N-tetraose |
| | galactosylated lacto-N-neotetraose |
| | glucosylated lacto-N-neotetraose |
| | N-acetylglucosaminylated lacto-N-neotetraose |
| | para-lacto-N-hexaose |
| | para-lacto-N-neohexaose |
| di-fucosylated | L-fucose |
| tetrasaccharides: | glucose |
| 2′3-difucosyllactose (DFL) | galactose |
| | fucosylated galactose |
| | fucosylated glucose |
| | lactose |
| | 3-fucosyllactose |
| | 2′-fucosyllactose |
| fucosylated | L-fucose |
| pentasaccharides: | glucose |
| lacto-N-fucopentaose I | galactose |
| (LNFP-I) | N-acetylglucosamine |
| or | lactose |
| lacto-N-fucopentaose Ii | fucosylated galactose |
| (LNFP-II) | fucosylated glucose |
| or | fucosylated N-acetylglucosamine |
| lacto-N-fucopentaose III | glucosylated galactose |
| (LNFP-III) | galactosylated galactose |
| or | N-acetylglucosaminylated galactose |
| lacto-N-fucopentaose V | galactosylated lactose |
| (LNFP-V) | glucosylated lactose |
| or | lacto-N-triose II |
| lacto-N-fucopentaose VI | 3-fucosyllactose |
| (LNFP-VI) | 2′-fucosyllactose |
| or | fucosylated lacto-N-triose II |
| lacto-N-neofucopentaose | difucosyllactose |
| (LNnFP) | lacto-N-neotetraose |
| | lacto-N-tetraose |
| | galactosylated lacto-N-tetraose |
| | glucosylated lacto-N-tetraose |
| | N-acetylglucosaminylated lacto-N-tetraose |
| | galactosylated lacto-N-neotetraose |
| | glucosylated lacto-N-neotetraose |
| | N-acetylglucosaminylated lacto-N-neotetraose |
| | para-lacto-N-hexaose |
| | para-lacto-N-neohexaose |
| | lacto-N-fucopentaose I |
| | lacto-N-fucopentaose II |
| | lacto-N-fucopentaose IIII |
| | lacto-N-fucopentaose V |
| | lacto-N-fucopentaose VI |
| | lacto-N-neofucopentaose |
| | mono-fucosylation of LNT or LNnT not leading to |
| | LNFP-I/-II/-III/-V/-VI or LNnFP |
| | di-fucosylation of LNT or LNnT |
| sialylated | N-acetylglucosamine |
| pentasaccharides: | N-acetylmannosamine |
| sialyllacto-N-tetraose a | N-acetylneuraminic acid |
| (LST-a) | glucose |
| or | galactose |
| sialyllacto-N-tetraose b | lactose |
| (LST-b) | sialylated galactose |
| or | sialylated glucose |
| sialyllacto-N-tetraose c | sialylated N-acetylglucosamine |
| (LST-c) | sialylated N-acetylmannosamine |
| | 6′-sialyllactose |
| | 3′-sialyllactose |
| | 3-sialyllactose |
| | 6-sialyllactose |
| | disialyllactose |
| | KDO-lactose |
| | lacto-N-triose II |
| | galactosylated lactose |
| | glucosylated lactose |
| | sialylated lacto-N-triose II |
| | lacto-N-tetraose |
| | lacto-N-neotetraose |
| | KDO-lacto-N-tetraose |
| | KDO-lacto-N-neotetraose |
| | galactosylated lacto-N-tetraose |
| | glucosylated lacto-N-tetraose |
| | N-acetylglucosaminylated lacto-N-tetraose |
| | galactosylated lacto-N-neotetraose |
| | glucosylated lacto-N-neotetraose |
| | N-acetylglucosaminylated lacto-N-neotetraose |
| | para-lacto-N-hexaose |
| | para-lacto-N-neohexaose |
| | sialyllacto-N-tetraose a |
| | sialyllacto-N-tetraose b |
| | sialyllacto-N-tetraose c |
| | di-sialylated lacto-N-tetraose |
| | di-sialylated lacto-N-neotetraose |
| | mono-sialylation of LNT or LNnT not leading to |
| | LST-a/-b/-c |
| di-fucosylated | L-fucose |
| hexasaccharides: | glucose |
| lacto-N-difucohexaose I | galactose |
| (LNDFH-I) | N-acetylglucosamine |
| or | lactose |
| lacto-N-difucohexaose II | fucosylated galactose |
| (LNDFH-II) | fucosylated glucose |
| | fucosylated N-acetylglucosamine |
| | glucosylated galactose |
| | galactosylated galactose |
| | N-acetylglucosaminylated galactose |
| | galactosylated lactose |
| | glucosylated lactose |
| | lacto-N-triose II |
| | 3-fucosyllactose |
| | 2′-fucosyllactose |
| | fucosylated lacto-N-triose II |
| | difucosyllactose |
| | lacto-N-tetraose |
| | galactosylated lacto-N-tetraose |
| | glucosylated lacto-N-tetraose |
| | N-acetylg lucosam inylated lacto-N-tetraose |
| | para-lacto-N-hexaose |
| | lacto-N-fucopentaose I |
| | lacto-N-fucopentaose II |
| | lacto-N-fucopentaose V |
| | di-fucosylation of LNT not leading to LNDFH-I/-II |
| sialylated | N-acetylglucosamine |
| hexasaccharides: | N-acetylmannosamine |
| disialyllacto-N-tetraose | N-acetylneuraminic acid |
| (DSLNT) | glucose |
| | galactose |
| | lactose |
| | sialylated galactose |
| | sialylated glucose |
| | sialylated N-acetylglucosamine |
| | sialylated N-acetylmannosamine |
| | 6′-sialyllactose |
| | 3′-sialyllactose |
| | 3-sialyllactose |
| | 6-sialyllactose |
| | disialyllactose |
| | KDO-lactose |
| | lacto-N-triose II |
| | galactosylated lactose |
| | glucosylated lactose |
| | sialylated lacto-N-triose II |
| | lacto-N-tetraose |
| | KDO-lacto-N-tetraose |
| | galactosylated lacto-N-tetraose |
| | glucosylated lacto-N-tetraose |
| | N-acetylglucosaminylated lacto-N-tetraose |
| | para-lacto-N-hexaose |
| | sialyllacto-N-tetraose a |
| | sialyllacto-N-tetraose b |
| | mono-sialylation of LNT or LNnT not leading to |
| | LST-a/-b/-c |
| | di-sialylation of lacto-N-tetraose not leading to |
| | DSLNT |

### SUMMARY

In a first aspect, disclosed is a method for the production of a desired oligosaccharide using a genetically-engineered microbial host cell that is able to produce the desired oligosaccharide, said microbial host cell expresses a heterologous glycosidase which is able to intracellularly degrade metabolic saccharide by-products that are generated during the intracellular biosynthesis of the desired oligosaccharide.

In a second aspect, disclosed is a genetically-engineered microbial host cell for the production of a desired oligosaccharide, wherein said microbial host cell is able to produce the desired oligosaccharide, and wherein said microbial host cell has been genetically-engineered to express a heterologous glycosidase which is able to intracellularly degrade metabolic saccharide by-products that are generated during the intracellular biosynthesis of the desired oligosaccharide.

In a third aspect, disclosed is the use of the genetically-engineered microbial host cell according to the second aspect for the production of a desired oligosaccharide.

In a fourth aspect, disclosed are oligosaccharides, i.e. desired oligosaccharides, that are produced by the method according to the first aspect and/or by using the genetically-engineered microbial host cell according to the second aspect.

In a fifth aspect, disclosed is the use of the desired oligosaccharides according to the fourth aspect for the production of a nutritional composition.

In a sixth aspect, disclosed are nutritional compositions containing a desired oligosaccharide according to the fourth aspect.

### BRIEF DESCRIPTION OF THE DRAWINGS

FIG. 1 shows a schematic representation of an embodiment of a microbial host cell expressing a heterologous glycosidase (e.g. an alpha-1,3-fucosidase) that is able to degrade metabolic saccharide by-products (e.g. 3-fucosyllactose and 2′3-difucosyllactose) that are generated during the intracellular biosynthesis of the desired oligosaccharide (2′-fucosyllactose), and wherein the microbial host cell is able to recycle the degradation products (e.g. fucose and lactose) resulting from the enzymatic activity of said glycosidase for the production of the desired oligosaccharide.

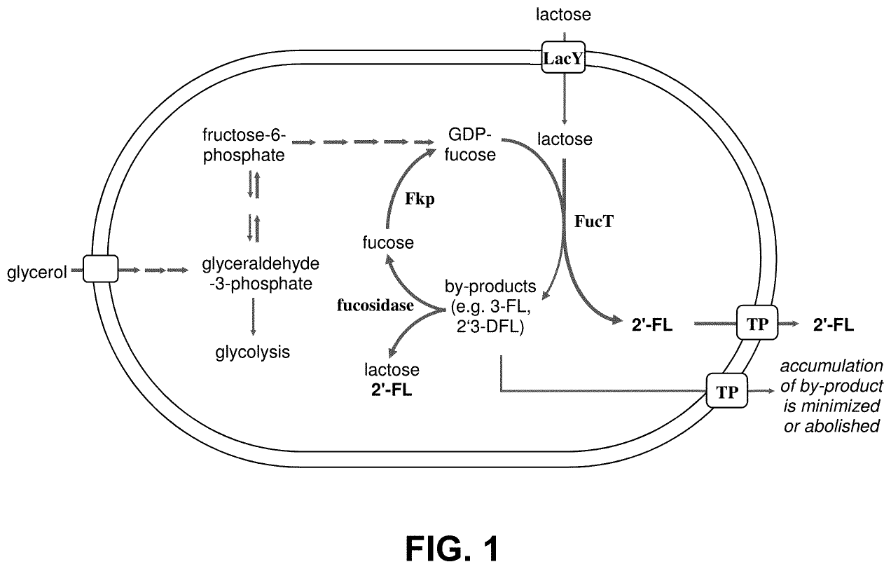

### DETAILED DESCRIPTION

According to the first aspect, provided is a method for the production of a desired oligosaccharide using a genetically-engineered microbial host cell, the method comprises the steps of:
(i) providing a genetically-engineered microbial host cell that is able to produce the desired oligosaccharide, wherein the microbial host cell has been genetically engineered to express a heterologous glycosidase which is able to intracellularly degrade metabolic saccharide by-products that are generated during the intracellular biosynthesis of the desired oligosaccharide, and wherein the microbial host cell is able to recycle degradation products resulting from the enzymatic activity of said glycosidase;
(ii) cultivating the genetically-engineered microbial host cell under conditions and in a medium permissive for the production of the desired oligosaccharide, thereby producing the desired oligosaccharide; and
(iii) optionally, recovering the desired oligosaccharide.

The term “desired” as used herein with respect to oligosaccharides refers to an oligosaccharide that is intended to be produced by the microbial host cell. The term “desired” is used to distinguish the oligosaccharide to be produced on purpose from other oligosaccharides the microbial host cell may produce. Said other oligosaccharides are considered to be “undesired”, regardless of whether or not these other oligosaccharides have a biological function, are involved in the biosynthesis of other cell compounds such as glycolipids, glycoproteins or polysaccharides, or are metabolic saccharide products that are generated during the intracellular biosynthesis of the desired oligosaccharide either due to subsidiary (undesired) enzymatic activities of one or more of the enzymes involved in the biosynthesis of the desired oligosaccharide, or due to the enzymatic activity of one or more enzymes which are not directly involved in the biosynthesis of the desired oligosaccharide but use an oligosaccharide as substrate which is generated as an intermediate in the metabolic pathway leading to the desired oligosaccharide.

The term “oligosaccharide” as used herein refers to a saccharide molecule consisting of three to twenty monosaccharide residues, wherein each of said monosaccharide residues in bound to at least one other of said monosaccharide units by a glycosidic linkage. The oligosaccharide may be a linear chain of monosaccharide residues or a branched chain of monosaccharide residues.

In an additional and/or alternative embodiment, the desired oligosaccharide is a human milk oligosaccharide (HMO).

In an additional and/or alternative embodiment, the desired oligosaccharide is a HMO selected from the group consisting of 2′-fucosyllactose (2′-FL), 3-fucosyllactose (3-FL), 2′3-difucosyllactose (DFL), lacto-N-triose II, lacto-N-tetraose (LNT), lacto-N-neotetraose (LNnT), lacto-N-fucopentaose I (LNFP-I), lacto-N-neofucopentaose I (LNnFP-I), lacto-N-fucopentaose II (LNFP-II), lacto-N-fucopentaose III (LNFP-III), lacto-N-fucopentaose V (LNFP-V), lacto-N-neofucopentaose V (LNnFPV), lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyllacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose (3-SL), 6′-sialyllactose (6-SL), sialyllacto-N-tetraose a (LST-a), sialyllacto-N-tetraose b (LST-b), sialyllacto-N-tetraose c (LST-c), and disialyllacto-N-tetraose.

The method comprises providing a genetically-engineered microbial host cell that is is able to produce the desired oligosaccharide.

The term “genetically-engineered” as used herein refers to the modification of the cell's genetic make-up using molecular biological methods. The modification of the cell's genetic make-up may include the transfer of genes within and/or across species boundaries, inserting, deleting, substituting and/or modifying nucleotides, triplets, genes, open reading frames, promoters, enhancers, terminators and other nucleotide sequences mediating and/or controlling gene expression. The modification of the cell's genetic make-up aims to generate a genetically modified organism possessing particular, desired properties. Genetically-engineered microbial host cell can contain one or more genes that are not present in the native (not genetically engineered) form of the cell. Techniques for introducing exogenous nucleic acid molecules and/or inserting exogenous nucleic acid molecules (recombinant, heterologous) into a cell's hereditary information for inserting, deleting or altering the nucleotide sequence of a cell's genetic information are known to the skilled artisan. Genetically-engineered cells can contain one or more genes that are present in the native form of the cell, wherein said genes are modified and re-introduced into the cell by artificial means. The term “genetically-engineered” also encompass cells that contain a nucleic acid molecule being endogenous to the cell, and that has been modified without removing the nucleic acid molecule from the cell. Such modifications include those obtained by gene replacement, site-specific mutations, and related techniques including those commonly referred to as “gene editing”.

The genetically-engineered microbial host cell may be a prokaryotic cell or a eukaryotic cell. Suitable microbial host cells include yeast cells, bacterial cells, archaebacterial cells and fungal cells.

In an additional and/or alternative embodiment, the prokaryotic cell is a bacterial cell, preferably a bacterial cell selected from bacteria of a genus selected from the group consisting of Bacillus, Bifidobacterium, Clostridium, Corynebacterium, Enterococcus, Lactobacillus, Lactococcus, Micrococcus, Micromonospora, Pseudomonas, Rhodococcus and Sporolactobacillus. Suitable bacterial species are Bacillus subtilis, B. licheniformis, B. coagulans, B. thermophilus, B. laterosporus, B. megaterium, B. mycoides, B. pumilus, B. lentus, B. cereus, B. circulans, Bifidobacterium longum, B. infantis, B. bifidum, Citrobacter freundii, Clostridium cellulolyticum, C. ljungdahlii, C. autoethanogenum, C. acetobutylicum, Corynebacterium glutamicum, Enterococcus faecium, E. thermophiles, Escherichia coli, Erwinia herbicola (Pantoea agglomerans), Lactobacillus acidophilus, L. salivarius, L. plantarum, L. helveticus, L. delbrueckii, L. rhamnosus, L. bulgaricus, L. crispatus, L. gasseri, L. casei, L. reuteri, L. jensenii, L. lactis, Pantoea citrea, Pectobacterium carotovorum, Proprionibacterium freudenreichii, Pseudomonas fluorescens, P. aeruginosa, Streptococcus thermophiles and Xanthomonas campestris.

In an additional and/or alternative embodiment, the eukaryotic cell is a yeast cell, preferably a yeast cell selected from the group consisting of Saccharomyces sp., in particular Saccharomyces cerevisiae, Saccharomycopsis sp., Pichia sp., in particular Pichia pastoris, Hansenula sp., Kluyveromyces sp., Yarrowia sp., Rhodotorula sp., and Schizosaccharomyces sp.

The genetically-engineered microbial host cell is able to produce the desired oligosaccharide. The term “able to produce” as used herein refers to the capability of the genetically-engineered microbial host cell to produce the desired oligosaccharide provided that the microbial host cell is cultivated under conditions and in a medium that are permissive for the microbial host cell to produce the desired oligosaccharide. Thus, the medium has to comprise a pH value in a defined range, a composition of ions and nutrients as well as of compounds required for maintaining viability and metabolic activity of the microbial host cell. If essential for the production of the desired oligosaccharide, the medium also has to contain sufficient amounts of any precursor required for biosynthesis of the desired oligosaccharide by the microbial host cell. Likewise, the conditions (e.g. temperature, pH, oxygen supply, agitation, supply of nutrients, etc.) for culturing the microbial host cell for producing the desired oligosaccharide have to be maintained such that the microbial host cell can be or remain metabolically active to produce the desired oligosaccharide.

In an additional and/or alternative embodiment, the genetically-engineered microbial host cell being able to produce a desired oligosaccharide is a microbial host cell that has been genetically engineered to be able to produce the desired oligosaccharide.

In an additional and/or alternative embodiment, the genetically-engineered microbial host cell has been genetically engineered to express a heterologous glycosyltransferase. The heterologous glycosidase is expressed in the genetically-engineered microbial host cell during fermentation, i.e. during the production or biosynthesis of the desired oligosaccharide. In an additional and/or alternative embodiment, expression of the heterologous glycosidase is constitutive in the genetically engineered microbial host.

The term “heterologous” as used herein refers to a nucleotide sequence, nucleic acid molecule or polypeptide that is foreign to a cell or organism, i.e. to a nucleotide sequence, nucleic acid molecule or polypeptide that does not naturally occur in said cell or organism. A “heterologous sequence” or a “heterologous nucleic acid” or “heterologous polypeptide”, as used herein, is one that originates from a source foreign to the particular host cell (e.g. from a different species), or, if from the same source, is modified from its original form. Thus, a heterologous nucleic acid operably linked to a promoter is from a source different from that from which the promoter was derived, or, if from the same source, is modified from its original form. The heterologous sequence may be stably introduced, e.g. by transfection, transformation, conjugation or transduction, into the genome of the host microbial host cell, thus representing a genetically modified host cell. Techniques may be applied which will depend on the host cell the sequence is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). Accordingly, a “heterologous polypeptide” is a polypeptide that does not naturally occur in the wild-type cell the genetically engineered cell is derived from, and a “heterologous glycosyltransferase” is a glycosyltransferase that does not naturally occur in the wild-type cell the genetically engineered cell is derived from.

In an additional and/or alternative embodiment, the heterologous glycosyltransferase is selected from the group consisting of fucosyltransferases, preferably α-1,2-fucosyltransferases and α-1,3-fucosyltransferases, glucosyltransferases, galactosyltransferases, preferably β-1,3-galactosyltransferases and β-1,4-galactosyltransferases, sialyltransferases, preferably α-2,3-sialyltransferases and α-2,6-sialyltransferases, and N-acetylglucosaminyltransferases.

Fucosyltransferases catalyze the transfer of fucose residues from the donor guanosine-diphosphate activated L-fucose (GDP-fucose) to several acceptor molecules. Fucosyltransferases are expressed in animals, plants, fungi and bacteria, and they are categorized according to the fucose linkage at the acceptor substrate. Therefore, α-1,2-, α-1,3/4- and α-1,6-fucosyltransferases are distinguished from each other. Suitable fucosyltransferases for heterologous expression in a genetically-engineered microbial host cell are disclosed—for example—in European patent application No. 17 180 176.

Sialyltransferases catalyze the transfer of N-acetylneuraminic acid (Neu5Ac) residues from the donor CMP-Neu5Ac to acceptor molecules. Sialyltransferases were found to be expressed in animals, plants, fungi and bacteria. Sialyltransferases are categorized according to the linkage that is formed between Neu5Ac and the acceptor molecule. Hence, α-2,3-, α-2,6- and α-2,8-sialyltransferases are distinguished from each other. Suitable sialyltransferases for heterologous expression in a genetically-engineered microbial host cell are disclosed—for example—in European patent application No. 17 183 391.

Galactosyltransferases catalyze the transfer of a galactose residue from the donor UDP-galactose to acceptor substrates. Galactosyltransferases are distinguished based on the linkage between the galactose and the acceptor molecule that is formed. Hence, β-1,3- and β-1,4-galactosyltransferases are distinguished from each other. A suitable β-1,3-galactosyltransferse for heterologous expression in a genetically-engineered microbial host cell is encoded by the Salmonella enterica wbdO gene. A suitable β-1,4-galctosyltransferse for heterologous expression in a genetically-engineered microbial host cell is encoded by the lexi gene of Aggregatibacter aphrophilus.

The genetically-engineered microbial host cell has been genetically engineered to express a heterologous glycosidase which is able to intracellularly degrade metabolic saccharide by-products that are generated during the intracellular biosynthesis of the desired oligosaccharide. Suitable glycosidases are glycosidases which are specific with respect to the glycosidic linkage that is hydrolyzed by the enzymatic activity and/or with respect to the substrate that is hydrolyzed by the glycosidase. Due to said specificity, the glycosidase hydrolyzes the undesired by-products, but not the desired oligosaccharide to be produced. In an additional and/or alternative embodiment, the glycosidase does not hydrolyze one or more of the precursors that are internalized or synthesized by the microbial host cell for producing the desired oligosaccharide. Preferably, the glycosidase is an exoglycosidase.

Exoglycosidases are glycoside hydrolase enzymes which break the glycosidic bonds at the terminal residue of an oligosaccharide structure.

In an additional and/or alternative embodiment, the heterologous glycosidase is selected from the group consisting of fucosidases including α-1,2-fucosidases and α-1,3-fucosidases, sialidases such as α-2,3-sialidases, α-2,6-sialidases, α-2,8-sialidases, galactosidases such as β-1,3-galactosidases, β-1,4-galactosidases and β-1,6-galactosidases, β-N-acetylhexosaminidases and glucosidases such as β-1,3-glucosidases.

A suitable fucosidase is an α-1,2-fucosidase. The α-1,2-fucosidase is a highly specific exoglycosidase that catalyzes the hydrolysis of linear alpha-1,2-linked L-fucopyranosyl residues from oligosaccharides. A preferred α-1,2-fucosidase is AfcA of Bifidobacterium bifidum (SEQ ID NO: 2).

In an additional and/or alternative embodiment, a genetically-engineered microbial host cell is provided that is able to produce 3-FL, wherein said genetically-engineered microbial host cell expresses an α-1,2-fucosidase. To be able to produce 3-FL, the genetically-engineered microbial host cell expresses an alpha-1,3-fucosyltransferase. Said alpha-1,3-fucosyltransferase is able to transfer a fucose residue from GDP-fucose to the glucose moiety of lactose as an acceptor substrate, thereby synthesizing 3-FL as desired oligosaccharide. 2′-FL and 2′3-DFL are undesired saccharide by-products in the production of 3-FL.

By expressing a heterologous α-1,2-fucosidase in the genetically-engineered microbial host cell that is able to produced 3-FL, production of the by-products 2′-FL and 2′3-DFL can be abolished or at least diminished in that these by-products are hydrolyzed within the genetically-engineered microbial host cell by the heterologous α-1,2-fucosidase. The resulting degradation products are fucose and lactose. Both, fucose and lactose, can be utilized by the genetically-engineered microbial host cell for the production of the desired 3-FL.

In an additional and/or alternative embodiment, the genetically-engineered microbial host cell has been genetically engineered to express the α-1,2-fucosidase. In an additional and/or alternative embodiment the genetically-engineered microbial host cell has been genetically engineered to contain a nucleic acid molecule comprising a nucleotide sequence encoding the α-1,2-fucosidase for its expression. Preferably, the nucleotide sequence encoding the α-1,2-fucosidase is a nucleotide sequence selected from the group consisting of
a nucleotide sequence as represented by SEQ ID NO: 1;
nucleotide sequences that are complementary to a nucleotide sequence which hybridizes to the nucleotide sequence as represented by SEQ ID NO: 1 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 1;
nucleotide sequences encoding a polypeptide having an amino acid sequence as represented by SEQ ID NO: 2; and
nucleotide sequences encoding a functional variant of the polypeptide sequences as represented by SEQ ID NO: 2, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 2.

The term “hybridize” or “hybridizing” as used herein means hybridizing under conventional conditions, as described in Sambrook et al. (1989) “Molecular Cloning, A Laboratory Manual” (Cold Spring Harbor Laboratory Press, New York), preferably under stringent conditions. Stringent hybridization conditions are for example: hybridizing in 4×SSC at 65° C. and subsequent multiple washing in 0.1×SSC at 65° C. for a total of about 1 hour. Less stringent hybridization conditions are for example: hybridizing in 4×SSC at 37° C. and subsequent multiple washing in 1×SSC at room temperature (about 21° C.). “Stringent hybridization conditions” can also mean: hybridizing at 68° C. in 0.25 M sodium phosphate, pH 7.2, 7% SDS, 1 mM EDTA and 1% BSA for 16 hours and followed by two washes with 2×SSC and 0 1% SDS at 68° C.

For expression of the nucleotide sequence encoding the α-1,2-fucosidase or functional variant thereof, said nucleotide sequence is operably linked to expression control sequences which mediate expression of the nucleotide sequence encoding the α-1,2-fucosidase or functional variant thereof in the genetically-engineered microbial host cell.

“Expression control sequences” are regulatory nucleotide sequences which are not part of the protein-encoding nucleotide sequence, but mediate the expression of protein-encoding nucleotide sequence. Regulatory element nucleotide sequences include promoters, cis regulatory elements, enhancers, introns and terminators. Depending on the type of regulatory element it is present on the nucleic acid molecule before the protein-coding nucleotide sequence (i.e. 3′ of) or behind the protein-encoding nucleotide sequence (i.e. 5′ of). The regulatory elements are functional in the microbial host cell.

The term “operably linked” means that a regulatory element is connected in such a way with the protein-encoding nucleotide sequence, i.e. is positioned in such a way relative to the protein-coding nucleotide sequence on, for example, a nucleic acid molecule that an expression of the protein-encoding nucleotide sequence under the control of the regulatory element can take place in a living cell.

For the purposes of the present invention, a “promoter” is an expression of a gene regulating nucleotide sequence, which is usually at the 5′end of a gene and via interaction with specific DNA-binding proteins mediates the initiation of transcription by RNA polymerase.

Furthermore, suitable promoters include synthetic promoters. These are promotors that have been created by molecular biology techniques that are not found in nature in this configuration. A synthetic promoter is a minimalistic promoter containing only one or more selected, defined cis-elements in addition to a minimal promoter. These cis-elements are binding sites for DNA-binding proteins such as transcription factors and are isolated from natural promoters, derived from previously isolated cis-elements, or produced technically by random recombination techniques and selected by appropriate methods; as compared with a natural promoter, due to its less complex construction a synthetic promoter is activated only by a few exogenous and endogenous factors and is therefore more specifically regulated.

The “minimal promoter” or “core”-promoter is a nucleotide sequence which contains the binding sites for the basal transcription factor complex and allows the accurate initiation of transcription by RNA polymerase II. Characteristic sequence motifs of the minimal promoter are the TATA box, the initiator element (Inr), the “TFBII recognition element” (BRE) and the “downstream core promoter element” (OPE). In the minimal promoter these elements can occur individually or in combination. The minimal promoter is or its sequence motifs are available, for example, from a bacterial, fungal or viral gene.

“Cis-elements” are nucleotide sequences that are located on the same nucleic acid molecule as the protein-encoding nucleotide sequence to be expressed. Cis-elements do not have to encode RNA or protein and in the direction of transcription can be located before or after the protein-encoding nucleotide sequence to be expressed. Cis-elements upstream before a protein-encoding nucleotide sequence to be expressed often provide necessary binding motifs in particular for transcription factors which engage as trans-acting elements (of Lat. trans, ‘beyond’), on the molecular level, from the other side in the regulation of the transcription of this gene. If, in addition, cis elements lead to an inhibition of the transcription, they are called silencers. Cis-elements that lead to an enhancement of the transcription are called enhancers. The totality of the cis/trans activities in the promoter determines the intensity with which the RNA polymerase carries out transcription.

Furthermore, a promoter may be a chimeric promoter and/or a promoter that has been modified by cis elements. The modification of a promoter can also mean the additional incorporation of a cis-element in the promoter which for example already has a cis-element naturally. Further, the modification also includes a multimerization of a cis element, in particular a multimerization of a naturally existing cis-element.

Compared with the native version such modified promoter may have altered properties with respect to specificity, expression level or background activity, for example.

Terminators are nucleotide sequences on the DNA, which usually mark the end of a gene and lead to the termination of transcription.

Another suitable fucosidase is a α-1,3-fucosidase. The α-1,3-fucosidase is a highly specific glycosidase that catalyzes the hydrolysis of α-1,3-linked L-fucopyranosyl residues from oligosaccharides. A preferred α-1,3-fucosidase is AfcB from Bifidobacterium bifidum (SEQ ID NO: 4).

In an additional and/or alternative embodiment, a genetically-engineered microbial host cell is provided that is able to produce 2′-FL, wherein said genetically-engineered microbial host organism expresses an α-1,3-fucosidase. To be able to produce 2′-FL, the genetically-engineered microbial host cell expresses an α-1,2-fucosyltranferase. Said alpha-1,2-fucosyltransferase is able to transfer a fucose residue from GDP-fucose to the galactose moiety of lactose as an acceptor substrate, thereby synthesizing 2′-FL as desired oligosaccharide. 3-FL and 2′3-DFL are undesired saccharide by-products in the production of 2′-FL.

By expressing a heterologous α-1,3-fucosidase in the genetically-engineered microbial host cell that is able to produce 2′-FL, production of the by-products 3-FL and 2′3-DFL can be abolished or at least diminished in that these by-products are hydrolyzed within the genetically-engineered microbial host cell by the heterologous α-1,3-fucosidase. The resulting degradation products are fucose and lactose. Both, fucose and lactose, can be utilized by the genetically-engineered microbial host organism for the production of the desired 2′-FL.

In an additional and/or alternative embodiment, the genetically-engineered microbial host cell has been genetically engineered to express the α-1,3-fucosidase. In an additional and/or alternative embodiment, the genetically-engineered microbial host cell has been genetically engineered to contain a nucleic acid molecule comprising a nucleotide sequence encoding the α-1,3-fucosidase for its expression. Preferably, the nucleotide sequence encoding the α-1,3-fucosidase is a nucleotide sequence selected from the group consisting of
a nucleotide sequence as represented by SEQ ID NO: 3;
nucleotide sequences that are complementary to a nucleotide sequence which hybridizes to the nucleotide sequence as represented by SEQ ID NO: 3 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 3;
nucleotide sequences encoding a polypeptide having an amino acid sequence as represented by SEQ ID NO: 4; and
nucleotide sequences encoding a functional variant of the polypeptide sequences as represented by SEQ ID NO: 4, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 4.

For expression of the nucleotide sequence encoding the α-1,3-fucosidase or functional variant thereof, said nucleotide sequence is operably linked to expression control sequences which mediate expression of the nucleotide sequence encoding the α-1,3-fucosidase or functional variant thereof in the genetically-engineered microbial host cell.

In an additional and/or alternative embodiment, a genetically engineered-microbial host cell is provided that is able to produce LNFP-I, wherein said genetically-engineered microbial host cell expresses an α-1,3-fucosidase. To be able to produce LNFP-I, the genetically-engineered microbial host cell expresses a β-1,3-N-acetylglucosaminylransferase, a β-1,3-galactosyltransferase and an α-1,2-fucosyltransferase. Said β-1,3-N-acetylglucosaminylransferase is able to transfer a GlcNAc residue from UDP-GlcNAc to the galactose moiety of lactose, thereby synthesizing lacto-N-triose-II (LNT-II). Said β-1,3-galactosyltransferase is able to transfer a galactose residue from UDP-galactose to the GlcNAc moiety of LNT-II, thereby synthesizing lacto-N-tetraose (LNT). Said α-1,2-fucosyltransferase is able to transfer a fucose residue from GDP-fucose to the terminal galactose moiety of LNT, thereby synthesizing LNFP-I. 3-FL and 2′3-DFL would be undesired by-products in the production of LNFP-I. By expressing an α-1,3-fucosidase in the genetically-engineered microbial host cell being able to produce LNFP-I, production of the by-products 3-FL and 2′3-DFL can be abolished or at least diminished in that these by-products are hydrolyzed by the α-1,3-fucosidase within the genetically-engineered microbial host cell. The resulting degradation products are fucose, lactose, and 2′-FL. Fucose and lactose can be utilized by the genetically-engineered microbial host organism for the production of the desired LNFP-I.

In an additional and/or alternative embodiment, the genetically-engineered microbial host cell has been genetically engineered to express the α-1,3-fucosidase. In an additional and/or alternative embodiment, the genetically-engineered microbial host cell has been genetically engineered to contain a nucleic acid molecule comprising a nucleotide sequence encoding the α-1,3-fucosidase for its expression. Preferably, the nucleotide sequence encoding the α-1,3-fucosidase is a nucleotide sequence selected from the group consisting of
a nucleotide sequence as represented by SEQ ID NO: 3;
nucleotide sequences that are complementary to a nucleotide sequence which hybridizes to the nucleotide sequence as represented by SEQ ID NO: 3 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 3;
nucleotide sequences encoding a polypeptide having an amino acid sequence as represented by SEQ ID NO: 4; and
nucleotide sequences encoding a functional variant of the polypeptide sequences as represented by SEQ ID NO: 4, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 4.

For expression of the nucleotide sequence encoding the α-1,3-fucosidase or functional variant thereof, said nucleotide sequence is operably linked to expression control sequences which mediate expression of the nucleotide sequence encoding the α-1,3-fucosidase or functional variant thereof in the genetically-engineered microbial host cell.

A suitable sialidase is an α-2,3-sialidase. The α-2,3-sialidase is a highly specific exoglycosidase that catalyzes the hydrolysis of linear α-2,3-linked L-sialyl residues from oligosaccharides. A preferred α-2,3-sialidase is NanB of Streptococcus pneumoniae (SEQ ID NO: 6).

In an additional and/or alternative embodiment, a genetically-engineered microbial host cell that is able to produce 6′-SL is provided, wherein said genetically-engineered microbial host cell expresses an α-2,3 sialidase. To be able to produce 6′-SL, the genetically engineered microbial host cell expresses an α-2,6-sialyltransferase. Said α-2,6-sialyltransferase is able to transfer a Neu5Ac residue from CMP-Neu5Ac to the galactose moiety of lactose as substrate, thereby synthesizing 6′-SL. 3′-SL is an undesired by-product in the production of 6′-SL.

By expressing an α-2,3-sialidase in the genetically-engineered microbial host cell that is able to produce 6′-SL, production of the by-product 3′-SL can be abolished or at least diminished in that this by-product is hydrolyzed by the α-2,3-sialidase within the genetically-modified microbial host cell. The resulting degradation products are N-acetylneuraminic acid and lactose. Both, N-acetylneuraminic acid and lactose, can be utilized by the genetically-engineered microbial host organism for producing the desired 6′-SL.

In an additional and/or alternative embodiment, the genetically-engineered microbial host cell has been genetically engineered to express the α-2,3-sialidase. In an additional and/or alternative embodiment, the genetically-engineered microbial host cell has been genetically engineered to contain a nucleic acid molecule comprising a nucleotide sequence encoding the α-2,3-sialidase for its expression. Preferably, the nucleotide sequence encoding the α-2,3-sialidase is a nucleotide sequence selected from the group consisting of
a nucleotide sequence as represented by SEQ ID NO: 5;
nucleotide sequences that are complementary to a nucleotide sequence which hybridizes to the nucleotide sequence as represented by SEQ ID NO: 5 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 5;
nucleotide sequences encoding a polypeptide having an amino acid sequence as represented by SEQ ID NO: 6; and
nucleotide sequences encoding a functional variant of the polypeptide sequences as represented by SEQ ID NO: 6, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 6.

For expression of the nucleotide sequence encoding the α-2,3-sialidase or functional variant thereof, said nucleotide sequence is operably linked to expression control sequences which mediate expression of the nucleotide sequence encoding the α-2,3-sialidase or functional variant thereof in the genetically-engineered microbial host cell.

A suitable galactosidase is a β-1,3-galactosidase. The β-1,3-galactosidase is an enzyme that catalyzes the hydrolysis of a β-1,3-linked galactose residue from oligosaccharides. A preferred β-1,3-galactosidase is Bga42A of Bifidobacterium longum (SEQ ID NO: 8).

In an additional and/or alternative embodiment, a genetically-engineered microbial host cell is provided that is able to produce LNnT, wherein said genetically-engineered microbial host cell expresses a β-1,3-galactosidase. To be able to produce LNnT, the genetically-engineered microbial host cell expresses a β-1,3-N-acetylglucosaminylransferase and a β-1,4-galactosyltransferase. Said β-1,3-N-acetylglucosaminylransferase is able to transfer a GlcNAc residue from UDP-GlcNAc to the galactose moiety of lactose, thereby synthesizing LNT-II. Said β-1,4-galactosyltransferase is able to transfer a galactose residue from UDP-galactose to the GlcNAc moiety of LNT-II, thereby synthesizing LNnT as desired oligosaccharide.

LNT is an undesired by-product in the production of LNnT. By expressing a β-1,3-galactosidase in the genetically-engineered microbial host cell being able to produce LNnT, production of the by-product LNT can be abolished or at least diminished in that this by-product is hydrolyzed within the genetically engineered microbial host cell by the heterologous β-1,3-galactosidase. The resulting degradation products are galactose and LNT-II. Galactose as well as LNT-II can be utilized by the genetically-engineered microbial host organism for the production of the desired LNnT.

In an additional and/or alternative embodiment, the genetically-engineered microbial host cell has been genetically engineered to express the β-1,3-galactosidase. In an additional and/or alternative embodiment the genetically-engineered microbial host cell has been genetically engineered to contain a nucleic acid molecule comprising a nucleotide sequence encoding the β-1,3-galactosidase for its expression.

In an additional and/or alternative embodiment, the genetically-engineered microbial host cell has been genetically engineered to express the β-1,3-galactosidase. In an additional and/or alternative embodiment the genetically-engineered microbial host cell has been genetically engineered to contain a nucleic acid molecule comprising a nucleotide sequence encoding the β-1,3-galactosidase for its expression. Preferably, the nucleotide sequence encoding the β-1,3-galactosidase is a nucleotide sequence selected from the group consisting of
a nucleotide sequence as represented by SEQ ID NO: 7;
nucleotide sequences that are complementary to a nucleotide sequence which hybridizes to the nucleotide sequence as represented by SEQ ID NO: 7 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 7;
nucleotide sequences encoding a polypeptide having an amino acid sequence as represented by SEQ ID NO: 8; and
nucleotide sequences encoding a functional variant of the polypeptide sequences as represented by SEQ ID NO: 8, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 8.

For expression of the nucleotide sequence encoding the β1,3-galactosidase or functional variant thereof, said nucleotide sequence is operably linked to expression control sequences which mediate expression of the nucleotide sequence encoding the β1,3-glucosidase or functional variant thereof in the genetically-engineered microbial host cell.

Another suitable galactosidase is a galactan β-1,3-galactosidase. The galactan β-1,3-galactosidase is an enzyme that catalyzes the hydrolysis of a β-1,3-linked galactose residue from galactose bearing oligosaccharide chains. A preferred galactan β-1,3-galactosidase is Ct1,3Gal43A of Clostridium thermocellum (SEQ ID NO: 10).

In an additional and/or alternative embodiment, the genetically-engineered microbial host cell has been genetically engineered to express the galactan β-1,3-galactosidase. In an additional and/or alternative embodiment the genetically-engineered microbial host cell has been genetically engineered to contain a nucleic acid molecule comprising a nucleotide sequence encoding the galactan β-1,3-galactosidase for its expression. Preferably, the nucleotide sequence encoding the galactan β-1,3-galactosidase is a nucleotide sequence selected from the group consisting of
a nucleotide sequence as represented by SEQ ID NO: 9;
nucleotide sequences that are complementary to a nucleotide sequence which hybridizes to the nucleotide sequence as represented by SEQ ID NO: 9 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 9;
nucleotide sequences encoding a polypeptide having an amino acid sequence as represented by SEQ ID NO: 10; and
nucleotide sequences encoding a functional variant of the polypeptide sequences as represented by SEQ ID NO: 10, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 10.

For expression of the nucleotide sequence encoding the galactan β-1,3-galactosidase or functional variant thereof, said nucleotide sequence is operably linked to expression control sequences which mediate expression of the nucleotide sequence encoding the galactan β-1,3-glucosidase or functional variant thereof in the genetically-engineered microbial host cell.

A suitable glucosidase is a β-1,3-glucosidase. The β-1,3-glucosidase is a highly specific exoglycosidase that catalyzes the hydrolysis of a β-1,3-linked glucose residue from oligosaccharides. A preferred β-1,3-glucosidase is PgIA of Paenibacillus sp. (SEQ ID NO: 12).

In an additional and/or alternative embodiment, a genetically-engineered microbial host organism is provided that is able to produce LNT or LNnT, wherein said genetically-engineered microbial host cell expresses a β1,3-glucosidase and/or a β-1,3-galactosidase. To be able to produce LNT, the genetically-engineered microbial host cell expresses a β-1,3-N-acetylglucosaminyltransferase and a β-1,3-galactosyltransferase. Said β-1,3-N-acetylglucosaminyltransferase is able to transfer a GlcNAc residue from UDP-GlcNAc to the galactose moiety of lactose, thereby synthesizing lacto-N-triose-II (LNT-II). Said β-1,3-galactosyltransferase is able to transfer a galactose residue from UDP-galactose to the GlcNAc moiety of LNT-II, thereby synthesizing lacto-N-tetraose (LNT). To be able to produce LNnT, the genetically-engineered microbial host cell expresses a β-1,3-N-acetylglucosaminyltransferase and a β-1,4-galactosyltransferase. Said β-1,3-N-acetylglucosaminyltransferase is able to synthesize LNT-II. Said a β-1,4-galactosyltransferase is able to transfer a galactose residue from UDP-galactose to the GlcNAc moiety of LNT-II, thereby synthesizing LNnT as desired oligosaccharide.

It is known to the skilled artisan that β-1,3-N-acetylglucosaminyltransferases like LgtA of Neisseria meningitidis accept a broad spectrum of donor substrates. While primarily transferring GlcNAc from UDP-GlcNAc to an appropriate acceptor saccharide, LgtA is also capable to use UDP-galactose or UDP-glucose as donor substrates. Using a genetically-engineered microbial host organism capable to produce LNT or LNnT as described, said β-1,3-N-acetylglucosaminyltransferase is also able to transfer a galactose residue from UDP-galactose as well as a glucose residue from UDP-glucose to the galactose moiety of lactose, thereby synthesizing the undesired by-products Gal(β1,3)Gal(β1,4)Glc and Glc(β1,3)Gal(β1,4)Glc, respectively.

By expressing a galactan β-1,3-galactosidase and/or a β-1,3-glucosidase in the genetically-engineered microbial host cell being able to produce LNT or LNnT, production of the by-products Gal(β1,3)Gal(β1,4)Glc and Glc(β1,3)Gal(β1,4)Glc can be abolished or at least diminished in that these by-products are hydrolyzed within the genetically-engineered microbial host cell by the galactan β-1,3-galactosidase and/or the β1,3-glucosidase. The resulting degradation products are galactose and/or glucose and lactose. Both monosaccharides as well as lactose can be utilized by the genetically-engineered microbial host cell to produce the desired LNT or LNnT.

In an additional and/or alternative embodiment, the genetically-engineered microbial host cell has been genetically engineered to express the β-1,3-glucosidase. In an additional and/or alternative embodiment, the genetically-engineered microbial host cell has been genetically engineered to contain a nucleic acid molecule comprising a nucleotide sequence encoding the β-1,3-glucosidase for its expression. Preferably, the nucleotide sequence encoding the β-1,3-glucosidase is a nucleotide sequence selected from the group consisting of
a nucleotide sequence as represented by SEQ ID NO: 11;
nucleotide sequences that are complementary to a nucleotide sequence which hybridizes to the nucleotide sequence as represented by SEQ ID NO: 11 under stringent conditions;
nucleotide sequences having a sequence identity of at least 70%, 75%, 80%, 85%, 90%, 95%, 96%, 97%, 98% or 99% to the nucleotide sequence as represented by SEQ ID NO: 11;
nucleotide sequences encoding a polypeptide having an amino acid sequence as represented by SEQ ID NO: 12; and
nucleotide sequences encoding a functional variant of the polypeptide sequences as represented by SEQ ID NO: 12, wherein the amino acid sequence of the functional variant has a sequence identity of at least 70%, 75%, 80%, 85%, 90%, 95%, 96%, 97%, 98% or 99% to the amino acid sequence as represented by SEQ ID NO: 10.

For expression of the nucleotide sequence encoding the β-1,3-glucosidase or functional variant thereof, said nucleotide sequence is operably linked to expression control sequences which mediate expression of the nucleotide sequence encoding the β-1,3-glucosidase or functional variant thereof in the genetically-engineered microbial host cell.

The genetically engineered microbial host cell is able to recycle at least one of the degradation products resulting from the enzymatic activity of the heterologous glycosidase in the genetically-engineered microbial host cell. Thus, the genetically-engineered microbial host cell can use at least one of the degradation products resulting from the enzymatic activity of the heterologous glycosidase for the production of the desired oligosaccharide. For example, a monosaccharide residue released from the undesired saccharide by-product by the heterologous glycosidase can be reactivated, i.e. bound to a nucleotide, to be transferred from the resulting nucleotide-activated monosaccharide to an acceptor substrate by a respective glycosyltransferase to give the desired oligosaccharide or a precursor of the desired oligosaccharide.

The method comprises the step of cultivating the genetically engineered microbial host cell in a medium that is permissive for the production of the desired oligosaccharide by said genetically engineered microbial host organism, and under conditions that are permissive for the production of the desired oligosaccharide by said genetically engineered microbial host organism.

The medium that is permissive for the production of the desired oligosaccharide by the genetically-engineered microbial host cell contains nutrients, at least one energy source, essential metals and minerals and a buffering agent. The medium optionally contains a precursor of the desired oligosaccharide, said precursor may be internalized by the genetically-engineered microbial host cell and utilized for the production of the desired oligosaccharide provided that the genetically-engineered microbial host cell is not able to synthesize said precursor on its own. Then, the genetically-engineered microbial host cell internalizes the precursor and subjects the precursor to the biosynthesis of the desired oligosaccharide. For example, lactose can be considered a precursor of 2′-fucosyllactose.

During the cultivation of the genetically-engineered microbial host cells for producing the desired oligosaccharide, permissive conditions are maintained. Conditions are “permissive” if the genetically-engineered microbial host cells that are cultured under these conditions stay alive and produce the desired oligosaccharide. Preferably the permissive culture conditions enable the genetically-engineered microbial host cells to multiply. Conditions that need to be kept at a certain value or within a certain range include pH, temperature, oxygen and concentrations of nutrients, energy sources and essential metals and minerals.

In an additional and/or alternative embodiment, the method comprises the step of recovering the desired oligosaccharide. The desired oligosaccharide may be recovered from the fermentation broth and/or from the genetically engineered microbial host organism.

The method as describe herein before is advantageous in that less or no undesired by-products are produced during the production of the desired oligosaccharide. Thereby, it is less cumbersome and costly to recover and purify the desired oligosaccharide from the fermentation broth or cell lysate.

In addition, much more substrate is specifically used for the production of the desired oligosaccharide instead of becoming inaccessible for the production of the desired oligosaccharide as it is incoporated in undesired by-products which can not be metabolized by the microbial host cell.

According to the second aspect, provided are genetically-engineered microbial host cells for the production of a desired oligosaccharide, wherein the microbial host cell is able to produce the desired oligosaccharide, and wherein the microbial host cell has been genetically engineered to express a heterologous glycosidase which is able to intracellularly degrade metabolic by-products that are generated during the intracellular biosynthesis of the desired oligosaccharide.

According to the third aspect, the genetically-engineered microbial host cells as described herein before are used for the production of a desired oligosaccharide. Using these genetically-engineered microbial host cells for the production of a desired oligosaccharide by fermentation is advantageous, because the production of undesired saccharide by-products is prevented or even abolished. Therefore, it saves resources and is less cumbersome to recover the desired oligosaccharide from the fermentation broth, as separation of the desired oligosaccharide from undesired oligosaccharide by-products can be avoided. Moreover, much more educt and energy-sources provided to the genetically-engineered microbial host cells according to the present invention are converted to the desired product as compared to a native microbial host cell that has not been genetically engineered to express a heterologous glycosidase.

According to the fourth aspect, the desired oligosaccharides that are produced by the method and/or the use of the genetically-engineered microbial host cells described herein before are preferably selected from the group of HMOs.

The desired oligosaccharides that are produced by the method and/or the use of the genetically-engineered microbial host cells described herein can be used for the production of a nutritional composition.

The nutritional composition is a medicinal composition, a dietary composition, an infant formula or the like.

The present invention will be described with respect to particular embodiments and with reference to drawings, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description and drawings provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

### Example 1: Metabolic Engineering of an E. coli BL21(DE3) Strain for the Production of 2′-Fucosyllactose

E. coli BL21(DE3) (Novagen) was used as parental strain for the construction of a host strain for the production of 2′-FL. Genetic engineering of the parental strain included gene disruption and deletion events and integration of heterologous genes.

Since 2′-fucosyllactose is synthesized from lactose, that is applied to the bacterial culture, and from GDP-L-fucose that is produced from the living cells, first the wild-type copy of the lacZgene encoding the endogenous β-galactosidase was inactivated by mutagenesis using mismatch oligonucleotides (Ellis et al., “High efficiency mutagenesis, repair, and engineering of chromosomal DNA using single-stranded oligonucleotides”, Proc. Natl. Acad. Sci. USA 98: 6742-6746 (2001)). Using the same method, the gene for the arabinose-isomerase araA was disrupted.

A lacZΩ gene fragment was introduced under the control of the temperature sensitive transcriptional repressor c1857. The lacZα fragment gene is expressed under the control of the E. coli BL21 (DE3) PgbA promoter in the strain, revealing a LacZ⁺ strain.

Genomic deletions were performed by λ Red mediated recombination according to the method of Datsenko and Warner (“One-step inactivation of chromosomal genes in Escherichia coli K-12 using PCR products”, Proc. Natl. Acad. Sci. USA 97:6640-6645 (2000)). The genes fucI and fucK, coding for the L-fucose isomerase and the L-fuculose kinase, respectively, have been deleted to prevent degradation of L-fucose. Also genes wzxC-wcaJ were deleted. WcaJ probably encodes a UDP-glucose: undecaprenyl phosphate glucose-1-phosphate transferase catalysing the first step in colanic acid synthesis (Stevenson et al., “Organization of the Escherichia coli K-12 gene cluster responsible for production of the extracellular polysaccharide colonic acid”, J. Bacteriol. 178:4885-4893; (1996)); production of colanic acid would compete for GDP-fucose with the fucosyltransferase reaction.

Genomic integration of heterologous genes was performed by transposition. Large gene clusters were integrated into the genome mediated by the hyperactive C9-mutant of the mariner transposase Himar1 (Lampe et al., “Hyperactive transposase mutants of the Himar1 mariner transposon”, Proc. Natl. Acad. Sci. USA 96:11428-11433 (1999)), that was inserted into the plasmid pEcomar under transcriptional control of the Pₐᵣₐ promotor. To enhance de novo synthesis of GDP-fucose, genes encoding phosphomannomutase (manB), mannose-1-phosphate guanosyltransferase (manC), GDP-mannose-4,6-dehydratase (gmd), and GDP-L-fucose synthase (wcaG) from E. coli K12 DH5a were overexpressed in the E. coli BL21(DE3) strain; the operon manCB was set under control of the constitutive promoter Pₜₑₜ, the operon gmd, wcaG is transcribed from the constitutive PT5 promoter. The transposon cassette <Pₜₑₜ-manCB-PT5-gmd, wcaG-FRT-dhfr-FRT> (SEQ ID NO: 13), including the gene for the dihydrofolate reductase for trimethoprim resistance, flanked by the inverted terminal repeats specifically recognized by the mariner-like element Himar1 transposase was inserted into the E. coli genome from pEcomar C9-manCB-gmd, wcaG-dhfr.

For chromosomal integration of single genes, the EZ-Tn5™ transposase (Epicentre, USA) was used. To produce EZ-Tn5 transposomes the gene of interest together with a FRT-site flanked antibiotic resistance cassette was amplified with primers that carried on both sites the 19-bp Mosaic End recognition sites (5′-CTGTCTCTTATACACATCT) for the EZ-Tn5 transposase. Using the EZ-Tn5™ transposase, the gene for the lactose importer LacY from E. coli K12 TG1 (acc. no. ABN72583), the 2-fucosyltransferase gene wbgL from E. coll:O126 (acc. no. ADN43847), and the gene yberc0001_9420 encoding a sugar efflux transporter of the major facilitator superfamily from Yersinia bercovieri ATCC 43970 (acc.no. EEQ08298) were integrated using the respective integration cassettes: <Pₜₑₜ-lacY-FRT-aadA-FRT> (SEQ ID NO: 14), <Pₜₑₜ-wbgLco-FRT-neo-FRT> (SEQ ID NO: 15), and <Pₜₑₜ-yberc0001_9420 co-FRT-cat-FRT> (SEQ ID NO: 16). The genes wbgL and yberc0001_9420 were synthetically synthesized and codon optimized (co) by GenScript Cooperation (USA). After successful integration of the lacY gene the resistance gene was eliminated from streptomycine resistant clones by the FLP recombinase encoded on plasmid pCP20 (Datsenko and Warner, “One-step inactivation of chromosomal genes in Escherichia coli K-12 using PCR products”, Proc. Natl. Acad. Sci. USA 97:6640-6645 (2000)).

Since E. coli BL21(DE3) lacks a functional gal-operon, a natively regulated copy of the galETKM operon from E. coli K was integrated into the B strain by EZ-transposition using integration cassette <P_{gal}-galE-galT-galK-galM> (SEQ ID NO: 17). Integrants were selected from MacConkey-agar containing 1% galactose as red colonies. The resulting strain is able to metabolize the monosaccharides glucose and galactose originating from lactose hydrolysis.

Further improvement concerning the synthesis of 2′-fucosyllactose by the E. coli strain was achieved by deletion of the pfkA gene, encoding the phosphofructokinase A. When cultivating E. coli on a gluconeogenic substrate like glycerol the phosphorylation of fructose-6-phosphate by PfkA is a highly ATP consuming treadmill reaction and, in addition, it competes with ManA for the substrate. The pfkA gene was deleted by homologous recombination according to Datsenko and Wanner (2000) using a gentamycin resistance cassette (aacC1) that was flanked by lox71/66 sites (Lambert, J M et al. (2007) Cre-lox-based system for multiple gene deletions and selectable-marker removal in Lactobacillus plantarum. Appl. Environ. Microbiol 73: 1126-1135). After successful deletion of the pfkA gene the antibiotic resistance gene was removed from E. coli genome using the Cre recombinase (Abremski, K et al. (1983) Studies on the properties of P1 site-specific recombination: evidence for topologically unlinked products following recombination. Cell 32: 1301-1311) that was cloned under the control of the Pₐᵣₐ promoter in the pKD46 (Datsenko and Wanner, 2000) chassis.

For different fucosyltransferases besides the transferase activity a GDP-L-fucose hydrolase activity was demonstrated. Also for wbgL, the α-1,2-fucosyltransferase used here for 2′-fucosyllactose synthesis this hydrolytic activity was shown (see EP 3 050 973 A1). To rescue free L-fucose for the 2′-fucosyllactose production and to eliminate the contaminating L-fucose from the culture broth, the fkp gene, encoding the bifunctional L-fucokinase/L-fucose 1-phosphat guanylyltranferase of Bacteroides fragilis, under transcriptional control of the Pₜₑₜ promoter, together with the lox71/66 flanked aacC1 gene was chromosomally integrated by transposition using the EZ-Tn5™ transposase, <Pₜₑₜ-fkp-lox-aacC1-lox> (SEQ ID NO: 18). After successful integration the gentamycin resistance gene was removed from the genome as described above.

To enhance the flux of the metabolized carbon source glycerol through the gluconeogenic pathway from triose-phosphates to fructose-6-phosphate to feed the GDPL-fucose biosynthesis the genes encoding the fructose-1,6-bisphosphate aldolase (fbaB) and a heterologous fructose-1,6-bisphosphate phosphatase (fbpase) from Pisum sativum were overexpressed. The fbaB gene from E. coli BL21 (DE3) was fused with the Pₜₑₜ promoter. The activity of the chloroplasic P. sativum FBPase is allosterically regulated by a disulfide-dithiol exchange due to reduction by thioredoxins. Exchange of the cysteine residue 153 to serine results in a constitutively active enzyme. The gene encoding the chloroplastic FBPase from P. sativum (acc. No. AAD10213) was purchased codon optimized for expression in E. coli, N-terminally tagged with a hexahistidine-tag and modified to encode the C153S variant of the enzyme from Genescript. The fbpase gene is transcribed from a T7 promoter. The cassette <Pₜₑₜ-fbaB-P_{T7}-His₆-fbpase-lox-aacC1-lox> (SEQ ID NO: 19) was used for EZ-Tn5™ transposase mediated integration in the host strain. After removal of the gentamycin resistance gene from the E. coli genome the strain was used for 2′-fucosyllactose production. Subsequently, this strain is named “strain A”.

### Example 2: Engineering of an E. coli BL21(DE3) Strain for the Production of 2′-Fucosyllactose at High Purity

Fed-batch cultivations using strain A for 2′-fucosyllactose production revealed the presence of by-products (3-fucosyllactose and 2′3-difucosyllactose) in the culture broth. In order to minimize the production of these by-products as well as for improving the carbon yield, an α-1,3-fucosidase was sub-cloned behind a constitutive promotor and integrated into the genome of strain A. Therefore, the afcB gene of Bifidobacterium bifidum (acc. No. AB474964) was fused with the constitutive P_{and} promoter and the gentamycin resistance gene. The resulting transposon cassette <P_{and}-afcB-lox-aacC1-lox> (SEQ ID NO: 20), flanked by the inverted terminal repeats specifically recognized by the mariner-like element Himar1 transposase, was inserted into the E. coli genome from pEcomar afcB-aacC1, generating “strain B”.

### Example 3: HPLC Analysis for the Detection of 2′-Fucosyllactose in Culture Supernatant

Analyses by HPLC were performed using a refractive index detector (RID-10A) (Shimadzu, Germany) and a Waters XBridge Amide Column 3.5 μm (250×4.6 mm) (Eschborn, Germany) connected to an HPLC system (Shimadzu, Germany). Elution was performed isocratically with 30% A: 50% (v/v) ACN in ddH₂O, 0.1% (v/v) NH₄OH and 70% B: 80% (v/v) ACN in ddH₂O, 0.1% (v/v) NH₄OH (v/v) as eluent at 35° C. and at a flow rate of 1.4 ml·min⁻¹. HPLC samples were sterile filtered (0.22 μm pore size) and cleared by solid phase extraction on an ion exchange matrix (Strata ABW, Phenomenex). 10 μl of the samples were applied to the column, and the 2′-fucosyllactose concentration was calculated according to a standard curve. Other sugars like L-fucose and/or other monosaccharides, lactose and/or other disaccharides, 3-fucosyllactose and/or other trisaccharides, 2′3-difucosyllactose and/or other tetrasaccharides as well as glycerol are also detectable using these analysis conditions. The relative amounts of detected sugars can be determined by comparing the AUC (area under the curve) of the all peaks in the chromatogram. Peaks also present in the water control are excluded from this calculation.

### Example 4: Production of 2′-Fucosyllactose in a Fermentative Process

Fermentations were conducted in 3 L-fermenters at 33° C. (New Brunswick, Edison, USA) starting with 1000 mL mineral salts medium containing 3 g/L KH₂PO₄, 12 g/L K₂HPO₄, 5 g/L (NH₄)₂SO₄, 0.3 g/L citric acid, 2 g/L MgSO₄×TH₂O, 0.1 g/L NaCl and 0.015 g/L CaCl₂)×6.H₂O supplemented with 1 g/L trace element solution (54.4 g·L⁻¹ ammonium ferric citrate, 9.8 g/L MnCl₂×4.H₂O, 1.6 g/L CoCl₂×6.H₂O, 1 g/L CuCl₂×2.H₂O, 1.9 g/L H₃BO₃, 9 g/L ZnSO₄×7.H₂O, 1.1 g/L Na₂MoO₄×2.H₂O, 1.5 g/L Na₂SeO₃, 1.5 g/L NiSO₄×6.H₂O) and containing 2% (v/v) glycerol as carbon source, 60 mM lactose and the antibiotic kanamycin (25 μg/mL). Aeration was maintained at 3 L/min. Dissolved oxygen was maintained at 20-30% saturation by controlling the rate of agitation. The pH was maintained at 7.0 by adding 25% ammonia solution. Cultivation was started with a 2.5% (v/v) inoculum from a preculture grown in the same glycerol containing medium but lacking lactose. After leaving the batch phase, indicated by a rise in the dissolved oxygen level, glycerol feeding (60% (v/v), supplemented with 2 g/L MgSO₄×7.H₂O, 0.015 g/L CaCl₂×6.H₂O and 1 mL/L trace element solution) was carried out at flow rates of 7.0-8.0 mUh, referring to the starting volume. Lactose feeding (0.66 M) was conducted throughout the cultivation and was adjusted intuitively in order to realize a constant lactose supply in the culture broth. Lactose feeding was stopped towards the end of the fermentation and cultivation was continued until lactose was completely converted to 2′-fucosyllactose. At around 94 hours after seeding the fermenter a 2′-fucosyllactose titer in the cell medium of about 150 g/L was reached when using the strain described in example 1 (strain A). The 2′-fucosyllactose production strain, genetically modified as described in example 2 (strain B), was cultivated comparably and yielded as well a 2′-fucosyllactose titer of about 150 g/L. However, the amount of by-products was significantly lower than after cultivating strain A (Table 2). Whereas the saccharide content in the culture supernatant of strain A consisted only to 94.22% of 2′-fucosyllactose, it was increased by 5.50% in the culture supernatant of strain B, exposing a purity of 99.72%.

TABLE 2
Qualitative HPLC analysis of the relative amount of saccharides
detectable in the culture supernatants of strain A and strain
B after 94 hours of cultivation (n.d.: not detectable).

| | Purity [area percentage] | | |
|---|---|---|---|
| | 2′-fucosyllactose | 3-fucosyllactose | 2′3-difucosyllactose |
| Strain A | 94.22% | 1.01% | 4.77% |
| Strain B | 99.72% | n. d. | 0.28% |

1. A method for production of a desired oligosaccharide using a genetically-engineered microbial host cell, the method comprising:
(i) providing a genetically-engineered microbial host cell that is able to produce the desired oligosaccharide, wherein the microbial host cell has been genetically engineered to express at least one heterologous glycosidase which is able to intracellularly degrade metabolic saccharide by-products that are generated during the intracellular biosynthesis of the desired oligosaccharide, and wherein the microbial host cell is able to recycle the degradation products resulting from the enzymatic activity of said glycosidase for the production of the desired oligosaccharide;
(ii) cultivating the genetically-engineered microbial host cell under conditions and in a medium permissive for production of the desired oligosaccharide, thereby producing the desired oligosaccharide; and
(iii) optionally, recovering the desired oligosaccharide.

2. A genetically-engineered microbial host cell for producing a desired oligosaccharide, wherein said microbial host cell is
a) able to produce the desired oligosaccharide;
b) has been genetically engineered to express at least one heterologous glycosidase which is able to intracellularly degrade one or more metabolic saccharide by-products that are generated during the intracellular biosynthesis of the desired oligosaccharide; and
c) is able to recycle one or more degradation products resulting from the enzymatic activity of said glycosidase for production of the desired oligosaccharide.

3. The method according to claim 1 or a genetically-engineered host cell capable of use therewith, wherein the heterologous glycosidase is selected from the group consisting of fucosidases, sialidases, hexosaminidases, galactosidases and glucosidases.

4. The method of claim 1 or a genetically-engineered microbial host cell useful therewith, wherein the heterologous glycosidase is selected from the group consisting of α-1,2-fucosidases, α-1,3-fucosidases, α-2,3-sialidases, α-2,6-sialidases, α-2,8-sialidases, β-1,3-galactosidases, β-1,4-galactosidases, β-1,6-galactosidases, β-N-acetylhexosaminidases and β-1,3-glucosidases.

5. The method of claim 1 or a genetically-engineered microbial host cell useful therewith, wherein the genetically-engineered microbial host cell has been genetically engineered to express a heterologous glycosyltransferase, optionally as glycosyltransferase selected from the group consisting of fucosyltransferases, sialyltransferases, galactosyltransferases, N-acetyl-glucosaminyltransferases and glucosyltransferases.

6. The method of claim 1 or a genetically-engineered microbial host cell useful therewith, wherein the microbial host cell has been genetically engineered to express a heterologous α-1,3-fucosyltransferase and a heterologous α-1,2-fucosidase.

7. The method of claim 1 or a genetically-engineered microbial host cell useful therewith, wherein the microbial host cell has been genetically engineered to express a heterologous α-1,2-fucosyltransferase and a heterologous α-1,3-fucosidase.

8. The method of claim 1 or a genetically-engineered microbial host cell useful therewith, wherein the microbial host cell has been genetically engineered to express a heterologous β-1,3-N-acetylglucosaminyltransferase, a heterologous α-1,2-fucosyltransferase, a heterologous β-1,3-galactosyltransferase and a heterologous α-1,3-fucosidase.

9. The method of claim 1 or a genetically-engineered microbial host cell useful therewith, wherein the microbial host cell has been genetically engineered to express a heterologous α-2,6-sialyltransferase and a heterologous α-2,3-sialidase.

10. The method of claim 1 or a genetically-engineered microbial host cell useful therewith, wherein the microbial host cell has been genetically engineered to express a heterologous β-1,3-N-acetylglucosaminyltransferase, a heterologous β-1,4-galactosyltransferase and a heterologous β-1,3-galactosidase and/or β-1,3-glucosidase and/or galactan-β-1,3-galactosidase.

11. The method of claim 1 or a genetically-engineered microbial host cell useful therewith, wherein the microbial host cell has been genetically engineered to express a heterologous β-1,3-N-acetylglucosaminyltransferase, a heterologous β-1,3-galactosyltransferase and a heterologous β-1,3-glucosidase and/or a galactan-β-1,3-galactosidase.

12. The method of claim 1 or a genetically-engineered microbial host cell useful therewith, wherein the desired oligosaccharide is a human milk oligosaccharide, optionally a human milk oligosaccharide selected from the group consisting of 2′-fucosyllactose (2′-FL), 3-fucosyllactose (3-FL), 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucosylhexaose II, para-Lacto-N-fucosylhexaose, fucosyl-lacto-N-sialylpentaose b, fucosyl-lacto-N-sialylpentaose c, fucosyl-lacto-N-sialylpentaose c, disialyl-lacto-N-fucopentaose, 3-fucosyl-3′-sialyllactose, 3-fucosyl-6′-sialyllactose, lacto-N-neodifucohexaose I, 3′-sialyllactose, 6′-sialyllactose, sialyllacto-N-tetraose a (LST-a), sialyllacto-N-tetraose b (LST-b), sialyllacto-N-tetraose c (LST-c), and disialyllacto-N-tetraose.

13. A product comprising the genetically-engineered microbial host cell according to claim 2 for production of a desired oligosaccharide, optionally an oligosaccharide selected from the group consisting of HMOs.

14. An oligosaccharide, optionally an oligosaccharide selected from the group consisting of HMOs, produced by the method according to claim 1 or a genetically engineered microbial host cell useful therewith, for manufacture of a nutritional composition.

15. A nutritional composition comprising at least one oligosaccharide that has been produced by the method of claim 1 or by use of a genetically-engineered microbial host cell useful therewith, wherein the at least one oligosaccharide is optionally a HMO.
